(19) United States
(12) Patent Application Publication
(10) Pub. No.: US 2017/0236648 A1
(43) Pub. Date: Aug. 17, 2017

(54) GRID CAPACITIVE POWER STORAGE SYSTEM

(71) Applicants: Pavel Ivan Lazarev, Menlo Park, CA (US); Ian S.G. Kelly-Morgan, San Francisco, CA (US); Mathew R. Robinson, San Francisco, CA (US); Paul Furuta, Sunnyvale, CA (US); Barry K. Sharp, Redwood City, CA (US)

(72) Inventors: Pavel Ivan Lazarev, Menlo Park, CA (US); Ian S.G. Kelly-Morgan, San Francisco, CA (US); Mathew R. Robinson, San Francisco, CA (US); Paul Furuta, Sunnyvale, CA (US); Barry K. Sharp, Redwood City, CA (US)

(73) Assignee: Capacitor Sciences Incorporated, Menlo Park, CA (US)

(21) Appl. No.: 15/430,391

(22) Filed: Feb. 10, 2017

Related U.S. Application Data
(63) Continuation-in-part of application No. 15/043,186, filed on Feb. 12, 2016.
Application No. 15/043,186 is a continuation-in-part of application No. 15/043,209, filed on Feb. 12, 2016.
Application No. 15/043,209 is a continuation-in-part of application No. 15/043,247, filed on Feb. 12, 2016.
Application No. 15/043,247 is a continuation-in-part of application No. 15/043,315, filed on Feb. 12, 2016.
(60) Provisional application No. 62/294,955, filed on Feb. 12, 2016.

(51) Int. Cl. H01G 9/07; H01G 9/04; C07D 471/06; C08F 220/18; C08F 220/34; H02J 7/00; C08G 69/32

(52) U.S. Cl. CPC H01G 9/07; H02J 7/0068; H02J 7/0042; H01G 9/04; C08G 69/32; C08F 220/18; C08F 220/34; C07D 471/06; H02J 7/345

(57) ABSTRACT

The present disclosure provides an energy storage system comprising at least one capacitive energy storage device and a DC-voltage conversion device. The capacitive energy storage device comprises at least one metacapacitor. The output voltage of the capacitive energy storage device is the input voltage of the DC-voltage conversion device. The capacitive energy storage system is capable of being charged from a power generation system and/or an electrical grid and discharging energy to a load and/or electrical grid. The capacitive energy storage system is configurable to supply external power as an operating power in a first state in which the external power is applied and/or to supply power as the operating power in a second state in which the external power is not applied.

### CLAIM OF PRIORITY

This application claims the benefit of U.S. Provisional Application No. 62/294,955 filed Feb. 12, 2016, which is hereby incorporated herein by reference in its entirety. This application is a continuation-in-part of U.S. patent applications Ser. Nos. 15/043,315, 15/043,186, 15/043,209, and 15/043,247, all of which were filed Feb. 12, 2016, the entire contents of all of which are incorporated herein by reference.

### FIELD OF THE DISCLOSURE

The present disclosure relates generally to an energy storage system to simultaneously enable multiple applications, a capacitive energy storage system, and more particularly to an energy storage cell comprising at least one capacitive energy storage device and a DC-voltage conversion device, a method of controlling a capacitive energy storage system, and a computer readable recording medium storing a program for executing the method.

### BACKGROUND

1. Description of the Related Technology

Interest in systems for storing energy and efficiently using the stored energy has increased to address problems such as environmental pollution and resource exhaustion. There is also increased interest in renewable energy that does not cause pollution during power generation. Thus, research into energy storage systems, which may be used with renewable energy, has been actively conducted as changes occur in domestic and overseas environments.

Many technical applications can benefit from rechargeable electrical energy storage. Most rechargeable electrical energy storage systems are based on rechargeable batteries. Rechargeable batteries store and release electrical energy through electrochemical reactions. Rechargeable batteries are used for automobile starters, portable consumer devices, light vehicles (such as motorized wheelchairs, golf carts, electric bicycles, and electric forklifts), tools, and uninterruptible power supplies. Emerging applications in hybrid internal combustion-battery and electric vehicles are driving the technology to reduce cost, weight, and size, and increase lifetime. Grid energy storage applications use rechargeable batteries for load-leveling, storing electric energy at times of low demand for use during peak periods, and for renewable energy uses, such as storing power generated from photovoltaic arrays during the day to be used at night. Load-leveling reduces the maximum power which a plant must be able to generate, reducing capital cost and the need for peaking power plants. Small rechargeable batteries are used to power portable electronic devices, power tools, appliances, and so on. Heavy-duty batteries are used to power electric vehicles, ranging from scooters to locomotives and ships. Rechargeable batteries are also used in distributed electricity generation and stand-alone power systems. Such applications often use rechargeable batteries in conjunction with a battery management system (BMS) that monitors battery parameters such as voltage, current, temperature, state of charge, and state of discharge and protects against operating the battery outside its safe operating area. Rechargeable batteries have drawbacks due to relatively large weight per unit energy stored, a tendency to self-discharge, susceptibility to damage if too deeply discharged, susceptibility to catastrophic failure if charged too deeply, limited power availability per unit weight, limited power availability per unit energy, relatively long charging times, and degradation of storage capacity as the number of charge-discharge cycles increases.

Alternatives to batteries for rechargeable energy storage include capacitor-based systems. Capacitors store energy in the form of an electrostatic field between a pair of electrodes separated by a dielectric layer. When a voltage is applied between two electrodes, an electric field is present in the dielectric layer. Unlike batteries, capacitors can be charged relatively quickly, can be deeply discharged without suffering damage, and can undergo a large number of charge discharge cycles without damage. Capacitors are also lower in weight than comparable batteries. Despite improvements in capacitor technology, including the development of ultracapacitors and supercapacitors, rechargeable batteries store more energy per unit volume. One drawback of capacitors compared to batteries is that the terminal voltage drops rapidly during discharge. By contrast, battery systems tend to have a terminal voltage that does not decline rapidly until nearly exhausted. Also, because the energy stored on a capacitor increases with the square of the voltage for linear dielectrics and at a power greater-than or equal to 2 for meta-dielectrics, capacitors for energy storage applications typically operate at much higher voltages than batteries. Furthermore, energy is lost if constant current mode is not used during charge and discharge. These characteristics complicate the design of power electronics for use with meta-capacitors and differentiate the meta-capacitor management system from battery management systems that are presently in use

It is within this context that aspects of the present disclosure arise.

### Introduction

Aspects of the present disclosure address problems with conventional rechargeable electrical energy storage technology by combining a capacitive energy storage device having one or more meta-capacitors with a DC-voltage conversion device having one or more switch mode voltage converters coupled to the terminals of the capacitive energy storage device. Meta-capacitors have greater energy storage capacity than conventional ultracapacitors or supercapacitors. The DC-voltage conversion device regulates the voltage on the capacitive energy storage device during charging and discharging.

A voltage conversion device typically includes a voltage source (an input), one or more active or passively controlled switches, one or more inductive elements (some advanced converters, e.g., charge-pump circuits, do not specifically use inductors per se though there may be parasitic inductance in the circuit board and/or wiring), one or more energy storage elements(e.g., capacitors and/or inductors), some way of sensing output voltage and/or current, and some way of controlling the switches to create a specific output voltage or current, and terminals to connect this device to external inputs and outputs such as various loads. A standard circuit for producing an output voltage Vₒᵤₜ that is less than the input voltage Vᵢₙ (Vₒᵤₜ/Vᵢₙ<1) is called a buck converter, and a standard circuit for producing an output voltage that is greater than the input voltage (Vₒᵤₜ/Vᵢₙ>1) is called a boost converter. The basic circuit often used to describe buck conversion is a switched LC filter (FIG. 1). The load can be thought of as a resistor that will vary its resistance to achieve a set current moving through it. Effectively, this is an LCR low-pass filter, with the capacitor and resistor in parallel. When the switch is closed, the LC network begins to absorb energy, and current begins to flow through the inductor. However, when the switch is opened while current is flowing, the inductor will attempt to maintain the current i(t) and will generate reverse voltage v(t) following equation (1).

$$v ( t ) = L \frac{di ( t )}{dt} , \qquad ( 1 )$$

The reverse voltage generated will be extremely high if the incremental change in current di occurs over a sufficiently short increment of time dt, and this may damage or destroy the switching element SW1. Therefore, it is necessary to provide a path to ground so that current can continue to flow. This path can be implemented with a diode that operates as a one-way valve, opening automatically when the inductor tries to pull current out of the switching element SW1 (see FIG. 2). This is called a non-synchronous buck converter, because the diode is automatically synchronized with the switching of a power transistor, such as a metal oxide semiconductor field effect transistor (MOSFET). Such a converter does not need to be actively synchronized. A possible issue with this type of circuit is that the turn-on voltage of the diode needs to be reached and maintained while the switching element SW1 is turned off and the diode is active. This means that there will always be a voltage drop of, e.g., ˜0.6V across the diode due to current flowing through it, and therefore a power loss. This can be improved by implementing a synchronous converter design, where the diode is replaced with a second switch SW2 (see FIG. 3) and an electronic controller actively synchronizes the activity of both switches such that they are never on at the same time.

The delay between turn-off and turn-on of the MOSFETs in a synchronous design needs to ensure that a shoot-through event does not occur. Although two separate pulses can be set up with a delay, a better solution would use only a single pulse width modulation (PWM) channel set up and would automatically derive the second signal. With a little bit of thought, this can be achieved using digital buffers (or inverters) to introduce a time delay into the switching signals applied to the switches SW1 and SW2 shown in FIG. 3. Typical gates have 2-10 ns propagation delay, but programmable logic devices such as a complex programmable logic device (CPLD) or field programmable gate array (FPGA) can be programmed with variable propagation delay. FIG. 4 demonstrates the signal treatment required to generate a pair of signals, S′ and ! S&&!S″ correspondingly to switches SW1, SW2 with the required time delay spacing, with the only inputs being a pulse-width modulated signal, S, and a time delay, t_{delay}. S′(t)=S(t+t_{delay}) and S″(t)=S(t+2*t_{delay}). In FIG. 4, it is assumed that a switch is “closed”, i.e., conducting, when the switching signal is high and “open”, i.e., non-conducting when the switching signal is low. In FIG. 4, S is an input PWM input signal. S′ is the input signal S delayed by t_{delay}. S″ is S′ delayed by 2*t_{delay}, !S is the inverse of the input signal S, !S″ is the inverse of signal S″, and !S&&!S″ is the logical AND of !S with !S″.

When deciding between synchronous or non-synchronous it is important to consider the efficiency losses due to switching (e.g., energy needed to move charge on and off the gate of a MOSFET) and those due to conduction through the diode. Synchronous converters tend to have an advantage in high-ratio conversion. They are also a fundamental building block of the split-pi-bidirectional converter because the extra switches are needed to provide dual-purpose buck or boost.

In the off-state, the boost converter delivers the supply voltage directly to the load through the second switch element SW2 in FIG. 5. The process of increasing the voltage to the load is started by opening the switching element SW2 and closing the switching element SW1 (FIG. 6). Due to the additional voltage drop on inductor L1, current flowing through inductor L1 will increase over time (see, equation (2)).

$$i ( t ) - i ( {t}_{0} ) = \frac{1}{L 1} {\int }_{{t}_{0}}^{t} v ( t ) dt , \qquad ( 2 )$$

When the circuit is returned to the “OFF” state, the inductor will attempt to maintain the same current that it had before by increasing its voltage drop proportional to the change in current (see, equation (3)).

$$v ( t ) = L 1 \frac{di ( t )}{dt} , \qquad 3 )$$

In the “off state” the switching element SW2 is closed so that this increased voltage gets translated to the output capacitor. The output capacitor provides filtering; averaging between Vᵢₙ and the inductor's voltage spikes.

N-channel MOSFET (NMOS), P-channel MOSFET (PMOS), and push-pull complementary metal oxide semiconductor (CMOS) topologies of a stacked MOSFET for fully integrated implementations in Honeywell's 150 nm SOI Radiation Hardened process described in following paper (Jennifer E et al., “High-Voltage Switching Circuit for Nanometer Scale CMOS Technologies” Manuscript received Apr. 30, 2007), which is incorporated herein by reference. The stacked MOSFET is a high-voltage switching circuit. A low-voltage input signal turns on the first MOSFET in a stack of MOSFET devices, and the entire stack of devices is turned on by charge injection through parasitic and inserted capacitances. Voltage division provides both static and dynamic voltage balancing, preventing any device in the circuit from exceeding its nominal operating voltage. The design equations for these topologies are presented. Simulations for a five device stack implemented in Honeywell's 150 nm process verify the static and dynamic voltage balancing of the output signal. The simulated stack is shown to handle five times the nominal operating voltage.

An example of a reliable circuit configuration for stacking power metal-oxide semiconductor field effect transistors (MOSFETs) is described, e.g., in R. J. Baker and B. P. Johnson, “Stacking Power MOSFETs for Use in High Speed Instrumentation”, Rev. Sci. Instrum., Vol. 63, No. 12, December 1992, pp. 799-801, which is incorporated herein by reference. The resulting circuit has a hold off voltage N times larger than a single power MOSFET, where N is the number of power MOSFETs used. The capability to switch higher voltages and thus greater amounts of power, into a 50 ohm load, in approximately the same time as a single device is realized. Design considerations are presented for selecting a power MOSFET. Using the design method presented, a 1.4 kV pulse generator, into SO 50 ohm, with a 2 ns rise time and negligible jitter is designed.

Another voltage switching circuit configuration is based on an Integrated Gate-Commutated Thyristor (IGCT). The integration of a 10-kV-IGCT and a fast diode in one press pack is an attractive solution for Medium Voltage Converters in a voltage range of 6 kV-7.2 kV if the converter power rating does not exceed about 5-6 MVA. (see, Sven Tschirley et al., “Design and Characteristics of Reverse Conducting 10-kV-IGCTs”, Proceedings of the 39th annual Power Electronics Specialist Conference, pages 92-98, 2008, which is incorporated herein by reference). Tschirley et al. describe the design and characterization of the world's first reverse conducting 68 mm 10-kV-IGCTs. On-state-, blocking and switching behavior of different IGCT and diode samples are investigated experimentally. The experimental results clearly show, that 10-kV-RC-IGCTs are an attractive power semiconductor for 6-7.2 kV Medium Voltage Converters.

Capacitors with high volumetric energy density, high operating temperature, low equivalent series resistance (ESR), and long lifetime are critical components for pulse-power, automotive, and industrial electronics. The physical characteristics of the dielectric material in the capacitor are the primary determining factors for the performance of a capacitor. Accordingly, improvements in one or more of the physical properties of the dielectric material in a capacitor can result in corresponding performance improvements in the capacitor component, usually resulting in performance and lifetime enhancements of the electronics system or product in which it is embedded. Since improvements in capacitor dielectric can directly influence product size, product reliability, and product efficiency, there is a high value associated with such improvements.

Compared to batteries, capacitors are able to store energy with very high power density, i.e. high charge/recharge rates, have long shelf life with little degradation, and can be charged and discharged (cycled) hundreds of thousands or millions of times. However, capacitors often do not store energy in as small a volume or weight as in case of a battery, or at low energy storage cost, which makes capacitors impractical for some applications, for example electric vehicles. Accordingly, it may be an advance in energy storage technology to provide capacitors of higher volumetric and mass energy storage density and lower cost.

### SUMMARY

Aspects of the present disclosure address problems with conventional rechargeable electrical energy storage technology by combining a capacitive energy storage device having one or more meta-capacitors (further described below) with a DC-voltage conversion device having one or more switch mode voltage converters coupled to the terminals of the capacitive energy storage device. Meta-capacitors have greater energy storage capacity than conventional ultracapacitors or supercapacitors. The DC-voltage conversion device regulates the voltage on the capacitive energy storage device during charging and discharging.

As used herein, a meta-capacitor is a dielectric film capacitor whose dielectric film is a meta-dielectric material, which is disposed between a first electrode and second electrode. In one embodiment, said electrodes are flat and planar and positioned parallel to each other. In another embodiment, the meta-capacitor comprises two rolled metal electrodes positioned parallel to each other. Additionally, a meta-dielectric material comprises of Sharp polymers and/or Furuta polymers.

The present disclosure provides an energy storage cell comprising a capacitive energy storage device having one or more meta-capacitors and a DC-voltage conversion device having one or more switch mode voltage converters. The power port (consisting of a positive terminal and a negative terminal, or anode and cathode) on the capacitive energy storage device is connected to the capacitor-side power port on the DC-voltage conversion device. The DC-voltage conversion device has one or more other power ports, which may interface to external circuitry. The power ports are intended to convey power with associated current and voltage commiserate to the specification for the cell. Each terminal in the port is a conductive interface. Each cell may include means to monitor and/or control parameters such as voltage, current, temperature, and other important aspects of the DC-voltage conversion device.

In one aspect, a capacitive energy storage module may include one or more individual capacitive energy storage cells and one or more power buses consisting of an interconnection system, wherein a power bus connects the power ports of the individual energy storage cells, in parallel or series, to create common module power ports consisting of common anode(s) and common cathode(s) of the capacitive energy storage module. The module may have additional sensors to monitor temperature, module power, voltage and current of the interconnection system, and may include a communication bus and/or communication bus protocol translator to convey these sensor values as well as the values from the individual cells.

In another aspect, a capacitive energy storage system may include one or more of the aforementioned capacitive energy storage modules, an interconnection system and a system control computer that monitors, processes, and controls all the values on the aforementioned communication bus.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWING

FIG. 1 schematically shows the buck conversion device based on the switched LC filter.

FIG. 2 schematically shows the non-synchronous buck conversion device.

FIG. 3 schematically shows the synchronous buck conversion device.

FIG. 4 demonstrates the signal treatment required to generate a pair of signals with the required time delay spacing.

FIG. 5 schematically shows a boost converter in an “on state”.

FIG. 6 schematically shows a boost converter in an “off state”.

FIG. 7A shows a capacitive energy storage device containing a single capacitive element connected to a two terminal port.

FIG. 7B shows an alternative configuration of a capacitive energy storage device containing multiple elements connected to a two terminal port.

FIG. 7C shows an alternative configuration of a capacitive energy storage device containing multiple elements connected to a two terminal port.

FIG. 7D shows an alternative configuration of a capacitive energy storage device containing multiple elements connected to a two terminal port.

FIG. 8A schematically shows a switch-mode voltage converter implementing a standard boost circuit.

FIG. 8B schematically shows a switch-mode voltage converter implementing a standard buck circuit.

FIG. 8C schematically shows a switch-mode voltage converter implementing a standard inverting buck/boost circuit.

FIG. 8D schematically shows a switch-mode voltage converter implementing a standard non-inverting and bi-directional buck/boost circuit.

FIG. 9A schematically shows a DC-voltage conversion device having two power ports and separate one or more boost and one or more buck converters for charging a meta-capacitor and separate one or more boost and one or more buck converters for discharging the meta-capacitor.

FIG. 9B schematically shows an alternative DC-voltage conversion device having two power ports and a one or more buck converters for charging a meta-capacitor and one or more buck boost converter for the discharging the meta-capacitor.

FIG. 9C schematically shows another alternative DC-voltage conversion device having two power ports and one or more boost converters for the charge and one or more buck converters for discharging a meta-capacitor.

FIG. 9D schematically shows another alternative DC-voltage conversion device having two power ports and one or more buck/boost converters for charging a meta-capacitor and one or more buck/boost converters for discharging the meta-capacitor.

FIG. 9E schematically shows yet another DC-voltage conversion device having two power ports and one or more bidirectional boost/buck converters for the charging and discharging a meta-capacitor.

FIG. 9F schematically shows still another DC-voltage conversion device having three power ports and separate one or more boost and one or more buck converters for charging a meta-capacitor and separate one or more boost and one or more buck converters for discharging the meta-capacitor.

FIG. 9G schematically shows another DC-voltage conversion device having three power ports and a one or more buck converters for charging a meta-capacitor and one or more buck boost converter for discharging the meta-capacitor.

FIG. 9H schematically shows another DC-voltage conversion device having three power ports and one or more buck/boost converters for charging a meta-capacitor and one or more buck/boost converters for discharging a meta-capacitor.

FIG. 9I schematically shows yet another DC-voltage conversion device having three power ports and one or more bidirectional boost/buck converters for the charging and discharging a meta-capacitor.

FIG. 10 schematically shows an energy storage cell according to aspects of the present disclosure.

FIG. 10A schematically shows a meta-capacitor with flat and planar electrodes according to aspects of the present disclosure.

FIG. 10B schematically shows a meta-capacitor with rolled (circular) electrodes according to aspects of the present disclosure.

FIG. 11 schematically shows an energy storage cell according to an alternative aspect of the present disclosure.

FIG. 12 schematically shows an energy storage cell according to an alternative aspect of the present disclosure.

FIG. 13A shows a constant voltage v_i(t) feeding the input of a converter and voltage v_c(t) on the capacitive energy storage device during charge as the converter transitions from buck to boost in accordance with aspects of the present disclosure.

FIG. 13B shows a constant voltage v_o(t) extracted from the output side of a converter and voltage v_c(t) on the capacitive energy storage device during discharge as the converter transitions from buck to boost in accordance with aspects of the present disclosure.

FIG. 14A shows a constant voltage v_i(t) feeding the input of a converter and voltage v_c(t) on the capacitive energy storage device during charge when v_{min,op}=v_i(t) in accordance with aspects of the present disclosure.

FIG. 14B shows a constant voltage v_o(t) extracted from the output side of a converter and voltage v_c(t) on the capacitive energy storage device during discharge when when v_{min,op}=v_i(t) in accordance with aspects of the present disclosure.

FIG. 15A shows an example of a single switch buck-boost converter that may be implemented in a switch-mode voltage converter, which could be selected for use in a DC voltage conversion device in an energy storage cell according to aspects of the present disclosure.

FIG. 15B shows an example of a four switch buck-boost converter that may be implemented in a switch-mode voltage converter, which could be selected for use in a DC voltage conversion device in an energy storage cell according to aspects of the present disclosure.

FIG. 16 shows an example of a capacitive energy storage module having two or more networked energy storage cells according to an alternative aspect of the present disclosure.

FIG. 17 shows an example of a capacitive energy storage system having two or more energy storage networked modules according to an alternative aspect of the present disclosure.

FIG. 18 shows a block diagram of the capacitive energy storage system with the possible connections to a power generation system, a load, and a grid, along with the necessary components for interacting with each.

FIG. 19 shows a flowchart illustrating a method of controlling the capacitive energy storage system's internal system power meter according to an aspect of the present disclosure.

FIG. 20 shows a flowchart illustrating a method of controlling the capacitive energy storage system's internal system power meter according to another aspect of the present disclosure.

FIG. 21 is a block diagram of a capacitive energy storage system (CESS), a CESS management system (CMS), and a power supply circuit that are coupled to each other, according to an aspect of the present disclosure.

FIG. 22 is a block diagram of one or more CESM racks according to an aspect of the present disclosure.

FIG. 23 is a circuit diagram illustrating a power supply circuit according to an aspect of the present disclosure.

FIG. 24 is a circuit diagram illustrating a power supply circuit according to another aspect of the present disclosure.

FIG. 25 is a circuit diagram illustrating a power supply circuit according to another aspect of the present disclosure.

FIG. 26 is a circuit diagram illustrating a power supply circuit according to another aspect of the present disclosure.

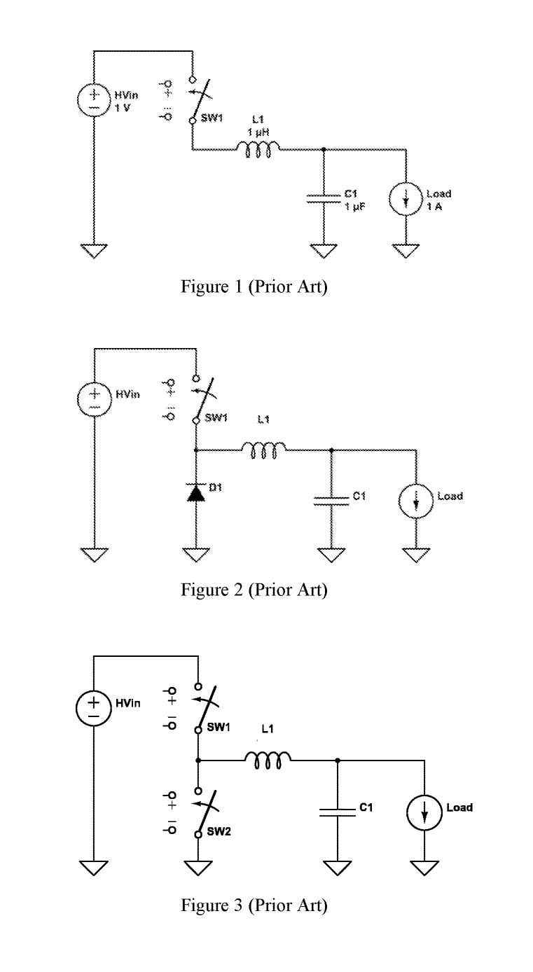

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

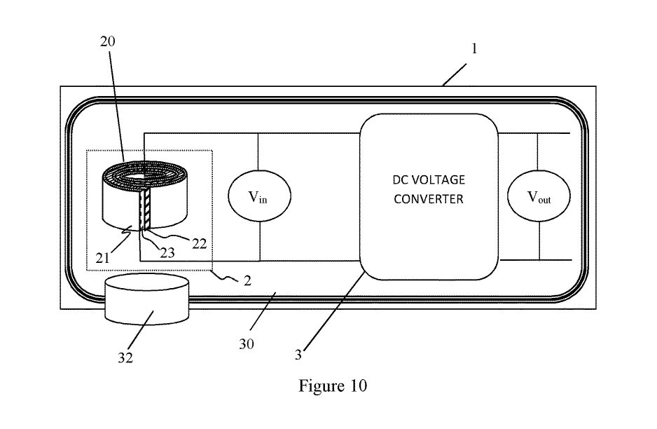

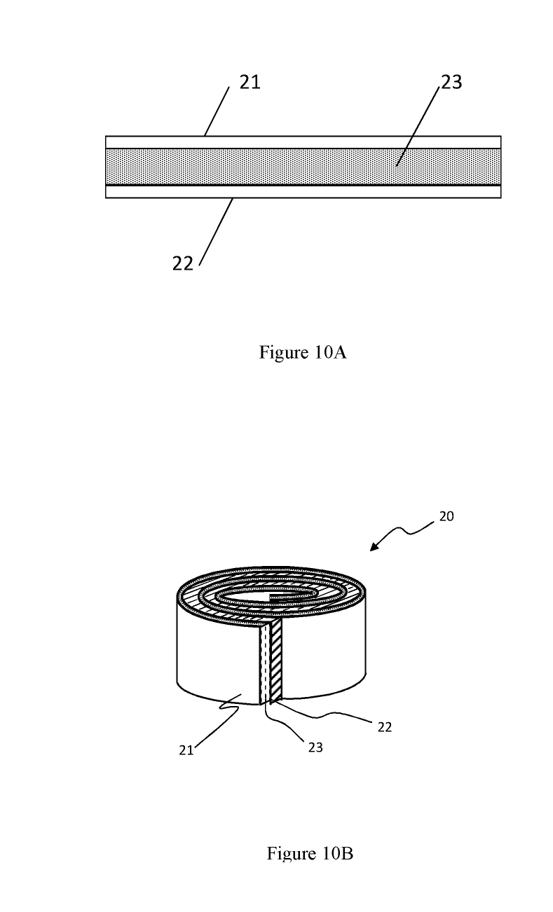

The present disclosure provides an energy storage cell comprising at least one capacitive energy storage device and a DC-voltage conversion device. FIG. 10 schematically shows a capacitive energy storage cell 1 comprising a capacitive energy storage device 2 that includes one or more meta-capacitors 20 and a DC-voltage conversion device 3, consisting of one more more switch-mode voltage converters 100, e.g. a buck converter, boost converter, buck/boost converter, bi-directional buck/boost (split-pi) converter, auk converter, SEPIC converter, inverting buck/boost converter, or four-switch buck/boost converter.

As used herein, a meta-capacitor is a capacitor comprising of a dielectric film that is a meta-dielectric material, which is disposed between a first electrode and second electrode. In one embodiment, said electrodes are flat and planar and positioned parallel to each other as shown in FIG. 10A. In another embodiment, the meta-capacitor comprises two rolled metal electrodes positioned parallel to each other as shown in FIG. 10B.

According to an aspect of the present disclosure a meta-capacitor may be configured as shown in FIG. 10A. The meta-capacitor comprises a first electrode 21, a second electrode 22, and a meta-dielectric layer 23 disposed between said first and second electrodes. The electrodes 21 and 22 may be made of a metal, such as copper, zinc, or aluminum or other conductive material and are generally planar in shape.

As used herein, a meta-capacitor is a capacitor comprising of a dielectric film that is a meta-dielectric material, which is disposed between a first electrode and second electrode. In one embodiment, said electrodes are flat and planar and positioned parallel to each other. In another embodiment, the meta-capacitor comprises two rolled metal electrodes positioned parallel to each other.

Said meta-dielectric materials are comprised of composite molecules having supra-structures formed from polymers. Examples of said polymers include so-called Sharp polymers and so-called Furuta co-polymers and so-called para-Furuta polymers as described in detail in commonly-assigned U.S. patent application Ser. No. 15/043,247 (Attorney Docket No. CSI-046 and Ser. No. 15/043,186 (Attorney Docket No. CSI-019A), and Ser. No. 15/043,209 (Attorney Docket No. CSI-019B), respectively, all filed Feb. 12, 2016, the entire contents of which are incorporated herein by reference.

Sharp polymers are composites of a polarizable core inside an envelope of hydrocarbon (saturated and/or unsaturated), fluorocarbon, chlorocarbon, siloxene, and/or polyethylene glycol as linear or branched chain oligomers covalently bonded to the polarizable core that act to insulate the polarizable cores from each other, which favorably allows discrete polarization of the cores with limited or no dissipation of the polarization moments in the cores. The polarizable core has hyperelectronic or ionic type polarizability. “Hyperelectronic polarization may be considered due to the pliant interaction of charge pairs of excitons, localized temporarily on long, highly polarizable molecules, with an external electric field.” (Roger D. Hartman and Herbert A. Pohl, “Hyper-electronic Polarization in Macromolecular Solids”, Journal of Polymer Science: Part A-1 Vol. 6, pp. 1135-1152 (1968)). Ionic type polarization can be achieved by limited mobility of ionic parts of the core molecular fragment.

A Sharp polymer has a general structural formula:

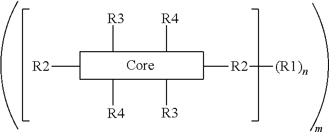

Where Core is an aromatic polycyclic conjugated molecule comprising rylene fragments. This molecule has flat anisometric form and self-assembles by pi-pi stacking in a column-like supramolecule. The substitute R1 provides solubility of the organic compound in a solvent. The parameter n is number of substitutes R1, which is equal to 0, 1, 2, 3, 4, 5, 6, 7 or 8. The substitute R2 is an electrically resistive substitute located in terminal positions, which provides resistivity to electric current and comprises hydrocarbon (saturated and/or unsaturated), fluorocarbon, siloxene, and/or polyethyleneglycol as linear or branched chains. The substitutes R3 and R4 are substitutes located on side (lateral) positions (terminal and/or bay positions) comprising one or more ionic groups from a class of ionic compounds that are used in ionic liquids connected to the aromatic polycyclic conjugated molecule (Core), either directly, e.g., with direct bound SP2-SP3 carbons, or via a connecting group. The parameter m is a number of the aromatic polycyclic conjugated molecules in the column-like supramolecule, which is in a range from 3 to 100,000.

In another implementation, the aromatic polycyclic conjugated molecule comprises an electro-conductive oligomer, such as a phenylene, thiophene, or polyacene quinine radical oligomer or combinations of two or more of these. In yet another embodiment of the composite organic compound, the electro-conductive oligomer is selected from phyenlyen, thiophene, or substituted and/or unsubstituted polyacene quinine radical oligomer of lengths ranging from 2 to 12 repeat units of the monomer forming the listed oligomer types or combination of two or more of these. Wherein the substitutions of ring hydrogens by O, S or NR5, and R5 is selected from the group consisting of unsubstituted or substituted C₁-C₁₈alkyl, unsubstituted or substituted C₂-C₁₈alkenyl, unsubstituted or substituted C₂-C₁₈alkynyl, and unsubstituted or substituted C₄-C₁₈ aryl.

In some implementations, the substitute providing solubility (R1) of the composite organic compound is C_{X}Q_{2X+1}, where X≧1 and Q is hydrogen (H), fluorine (F), or chlorine (Cl). In still another embodiment of the composite organic compound, the substitute providing solubility (R1) of the composite organic compound is independently selected from alkyl, aryl, substituted alkyl, substituted aryl, fluorinated alkyl, chlorinated alkyl, branched and complex alkyl, branched and complex fluorinated alkyl, branched and complex chlorinated alkyl groups, and any combination thereof, and wherein the alkyl group is selected from methyl, ethyl, propyl, butyl, iso-butyl and tent-butyl groups, and the aryl group is selected from phenyl, benzyl and naphthyl groups or siloxene, and/or polyethylene glycol as linear or branched chains.

In some implementations, at least one electrically resistive substitute (R2) of the composite organic compound is C_{X}Q_{2X+1}, where X≧1 and Q is hydrogen (H), fluorine (F), or chlorine (Cl). In another embodiment of the composite organic compound, at least one electrically resistive substitute (R2) is selected from the list comprising —(CH₂)ₙ—CH₃, —CH((CH—₂)ₙCH₃)₂) (where n≧1), alkyl, aryl, substituted alkyl, substituted aryl, branched alkyl, branched aryl, and any combination thereof and wherein the alkyl group is selected from methyl, ethyl, propyl, butyl, iso-butyl and tent-butyl groups, and the aryl group is selected from phenyl, benzyl and naphthyl groups. In yet another embodiment of the composite organic compound.

In some embodiments, the substitute R1 and/or R2 is connected to the aromatic polycyclic conjugated molecule (Core) via at least one connecting group. The at least one connecting group may be selected from the list comprising the following structures: ether, amine, ester, amide, substituted amide, alkenyl, alkynyl, sulfonyl, sulfonate, sulfonamide, or substituted sulfonamide.

In some embodiments, the substitute R3 and/or R4 may be connected to the aromatic polycyclic conjugated molecule (Core) via at least one connecting group. The at least one connecting group may be selected from the list comprising CH₂, CF₂, SiR₂O, CH₂CH₂O, wherein R is selected from the list comprising H, alkyl, and fluorine. In another embodiment of the composite organic compound, the one or more ionic groups include at least one ionic group selected from the list comprising [NR₄]⁺, [PR₄]⁺ as cation and [—CO₂]⁻, [—SO₃]⁻, [—SR₅]⁻, [—PO₃R]⁻, [—PR₅]⁻ as anion, wherein R is selected from the list comprising H, alkyl, and fluorine.

The present disclosure provides a Sharp polymer in the form of a composite organic compound. In one embodiment of the composite organic compound, the aromatic polycyclic conjugated molecule (Core) comprises rylene fragments. In another embodiment of the composite organic compound, the rylene fragments are selected from structures 1 to 21 as given in Table 1.

TABLE 1
Examples of the polycyclic organic molecule (Core) comprising rylene
fragments

| | |
|---|---|
| 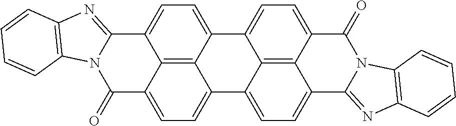 [SMILES: O=c1c2ccc3c4ccc5c6c(ccc(c7ccc(c2c37)c2nc3ccccc3n12)c46)c(=O)n1c2ccccc2nc51] | 1 |
| 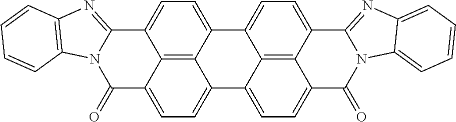 [SMILES: O=c1c2ccc3c4ccc5c(=O)n6c7ccccc7nc6c6ccc(c7ccc(c2c37)c2nc3ccccc3n12)c4c56] | 2 |
| 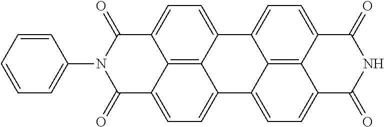 [SMILES: O=C1NC(=O)c2ccc3c4ccc5c6c(ccc(c7ccc1c2c73)c64)C(=O)N(c1ccccc1)C5=O] | 3 |
| 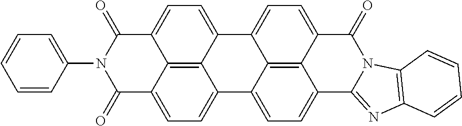 [SMILES: O=C1c2ccc3c4ccc5c(=O)n6c7ccccc7nc6c6ccc(c7ccc(c2c37)C(=O)N1c1ccccc1)c4c56] | 4 |
| 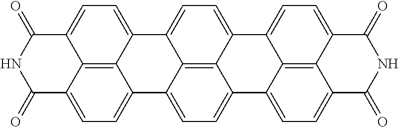 [SMILES: O=C1NC(=O)c2ccc3c4ccc5c6ccc7c8c(ccc(c9ccc(c%10ccc1c2c%103)c4c95)c86)C(=O)NC7=O] | 5 |
| 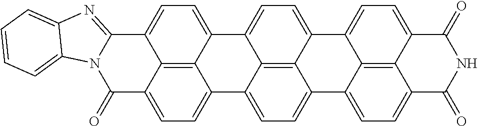 [SMILES: O=C1NC(=O)c2ccc3c4ccc5c6ccc7c8c(ccc(c9ccc(c%10ccc1c2c%103)c4c95)c68)c(=O)n1c2ccccc2nc71] | 6 |
| 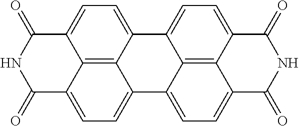 [SMILES: O=C1NC(=O)c2ccc3c4ccc5c6c(ccc(c7ccc1c2c73)c64)C(=O)NC5=O] | 7 |
| 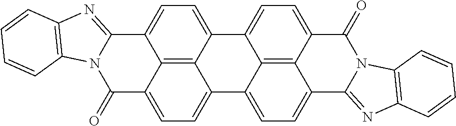 [SMILES: O=c1c2ccc3c4ccc5c6c(ccc(c7ccc(c2c37)c2nc3ccccc3n12)c46)c(=O)n1c2ccccc2nc51] | 8 |
| 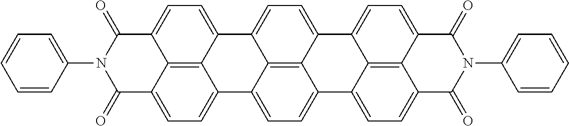 [SMILES: O=C1c2ccc3c4ccc5c6ccc7c8c(ccc(c9ccc(c%10ccc(c2c3%10)C(=O)N1c1ccccc1)c4c59)c86)C(=O)N(c1ccccc1)C7=O] | 9 |
| 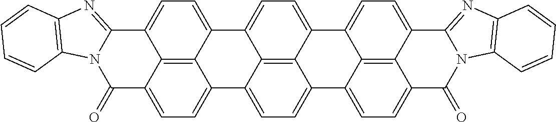 [SMILES: O=c1c2ccc3c4ccc5c6ccc7c(=O)n8c9ccccc9nc8c8ccc(c9ccc(c%10ccc(c2c3%10)c2nc3ccccc3n12)c4c59)c6c78] | 10 |
| 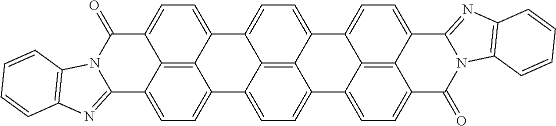 [SMILES: O=c1c2ccc3c4ccc5c6ccc7c8c(ccc(c9ccc(c%10ccc(c2c3%10)c2nc3ccccc3n12)c4c95)c68)c(=O)n1c2ccccc2nc71] | 11 |
| 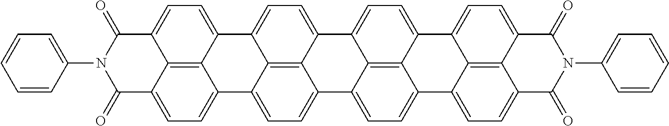 [SMILES: O=C1c2ccc3c4ccc5c6ccc7c8ccc9c%10c(ccc(c%11ccc(c%12ccc(c%13ccc(c2c3%13)C(=O)N1c1ccccc1)c4c5%12)c6c7%11)c%108)C(=O)N(c1ccccc1)C9=O] | 12 |
| 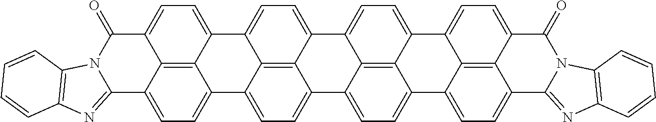 [SMILES: O=c1c2ccc3c4ccc5c6ccc7c8ccc9c(=O)n%10c%11ccccc%11nc%10c%10ccc(c%11ccc(c%12ccc(c%13ccc(c2c3%13)c2nc3ccccc3n12)c4c5%12)c6c7%11)c8c9%10] | 13 |
| 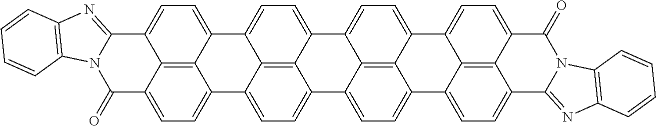 [SMILES: O=c1c2ccc3c4ccc5c6ccc7c8ccc9c%10c(ccc(c%11ccc(c%12ccc(c%13ccc(c2c3%13)c2nc3ccccc3n12)c4c5%12)c6c%117)c8%10)c(=O)n1c2ccccc2nc91] | 14 |
| 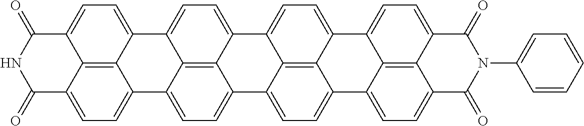 [SMILES: O=C1NC(=O)c2ccc3c4ccc5c6ccc7c8ccc9c%10c(ccc(c%11ccc(c%12ccc(c%13ccc1c2c%133)c4c%125)c6c%117)c%108)C(=O)N(c1ccccc1)C9=O] | 15 |
| 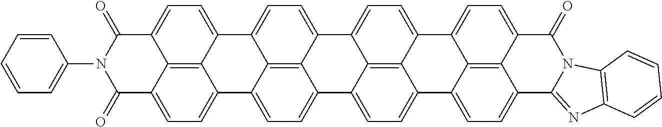 [SMILES: O=C1c2ccc3c4ccc5c6ccc7c8ccc9c(=O)n%10c%11ccccc%11nc%10c%10ccc(c%11ccc(c%12ccc(c%13ccc(c2c3%13)C(=O)N1c1ccccc1)c4c5%12)c6c7%11)c8c9%10] | 16 |
| 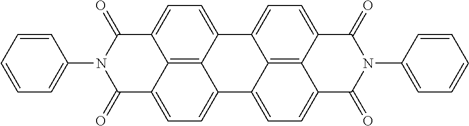 [SMILES: O=C1c2ccc3c4ccc5c6c(ccc(c7ccc(c2c37)C(=O)N1c1ccccc1)c64)C(=O)N(c1ccccc1)C5=O] | 17 |
| 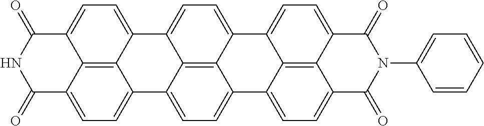 [SMILES: O=C1NC(=O)c2ccc3c4ccc5c6ccc7c8c(ccc(c9ccc(c%10ccc1c2c%103)c4c95)c86)C(=O)N(c1ccccc1)C7=O] | 18 |
| 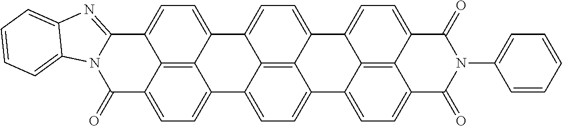 [SMILES: O=C1c2ccc3c4ccc5c6ccc7c(=O)n8c9ccccc9nc8c8ccc(c9ccc(c%10ccc(c2c3%10)C(=O)N1c1ccccc1)c4c59)c6c78] | 19 |
| 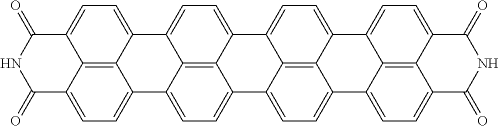 [SMILES: O=C1NC(=O)c2ccc3c4ccc5c6ccc7c8ccc9c%10c(ccc(c%11ccc(c%12ccc(c%13ccc1c2c%133)c4c%125)c6c%117)c%108)C(=O)NC9=O] | 20 |
| 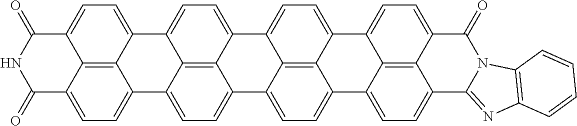 [SMILES: O=C1NC(=O)c2ccc3c4ccc5c6ccc7c8ccc9c%10c(ccc(c%11ccc(c%12ccc(c%13ccc1c2c%133)c4c%125)c6c%117)c8%10)c(=O)n1c2ccccc2nc91] | 21 |

In another embodiment of the composite organic compound, the aromatic polycyclic conjugated molecule comprises an electro-conductive oligomer, such as a phenylene, thiophene, or polyacene quinine radical oligomer or combinations of two or more of these. In yet another embodiment of the composite organic compound, the electro-conductive oligomer is selected from structures 22 to 30 as given in Table 2, wherein I=2, 3, 4, 5, 6, 7, 8, 9, 10, 11 or 12, Z is ═O, ═S or ═NR5, and R5 is selected from the group consisting of unsubstituted or substituted C₁-C₁₈alkyl, unsubstituted or substituted C₂-C₁₈alkenyl, unsubstituted or substituted C₂-C₁₈alkynyl, and unsubstituted or substituted C₄-C₁₈aryl:

TABLE 2
Examples of the polycyclic organic molecule (Core) comprising electro-
conductive oligomer

| | |
|---|---|
| 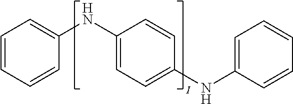 | 22 |
| 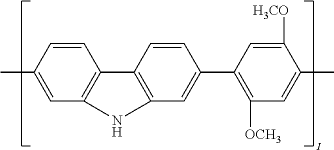 | 23 |
| 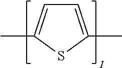 | 24 |
| 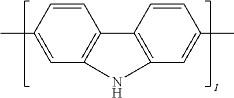 | 25 |
| 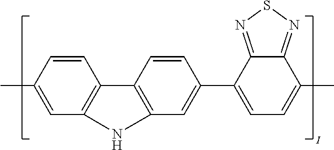 | 26 |
| 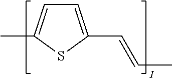 | 27 |
| 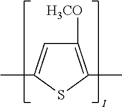 | 28 |
| 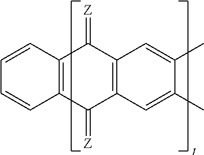 | 29 |
| 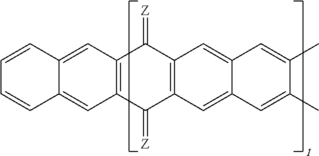 | 30 |

In some embodiments, the substitute providing solubility (R1) of the composite organic compound is C_{X}Q_{2X+1}, where X≧1 and Q is hydrogen (H), fluorine (F), or chlorine (Cl). In still another embodiment of the composite organic compound, the substitute providing solubility (R1) of the composite organic compound is independently selected from alkyl, aryl, substituted alkyl, substituted aryl, fluorinated alkyl, chlorinated alkyl, branched and complex alkyl, branched and complex fluorinated alkyl, branched and complex chlorinated alkyl groups, and any combination thereof, and wherein the alkyl group is selected from methyl, ethyl, propyl, butyl, iso-butyl and tent-butyl groups, and the aryl group is selected from phenyl, benzyl and naphthyl groups or siloxane, and/or polyethyleneglycol as linear or branched chains.

In one embodiment of the composite organic compound, the solvent is selected from benzene, toluene, xylenes, acetone, acetic acid, methylethylketone, hydrocarbons, chloroform, carbontetrachloride, methylenechloride, dichlorethane, chlorobenzene, alcohols, nitromethan, acetonitrile, dimethylforamide, 1,4-dioxane, tetrahydrofuran (THF), methylcyclohexane (MCH), and any combination thereof.

In some embodiments, at least one electrically resistive substitute (R2) of the composite organic compound is CXQ2X+1, where X≧1 and Q is hydrogen (H), fluorine (F), or chlorine (Cl). In another embodiment of the composite organic compound, at least one electrically resistive substitute (R2) is selected from the list comprising —(CH2)n-CH3, —CH((CH2)nCH3)2) (where n≧1), alkyl, aryl, substituted alkyl, substituted aryl, branched alkyl, branched aryl, and any combination thereof and wherein the alkyl group is selected from methyl, ethyl, propyl, butyl, I-butyl and t-butyl groups, and the aryl group is selected from phenyl, benzyl and naphthyl groups. In yet another embodiment of the composite organic compound.

In some embodiments, at least one electrically resistive substitute (R2) is selected from the group of alkyl, aryl, substituted alkyl, substituted aryl, fluorinated alkyl, chlorinated alkyl, branched and complex alkyl, branched and complex fluorinated alkyl, branched and complex chlorinated alkyl groups, and any combination thereof, and wherein the alkyl group is selected from methyl, ethyl, propyl, n-butyl, iso-butyl and tert-butyl groups, and the aryl group is selected from phenyl, benzyl and naphthyl groups or siloxane, and/or polyethyleneglycol as linear or branched chains.

In some embodiments, the substitute R1 and/or R2 is connected to the aromatic polycyclic conjugated molecule (Core) via at least one connecting group. The at least one connecting group may be selected from the list comprising the following structures: 31-41 as given in Table 3, where W is hydrogen (H) or an alkyl group.

TABLE 3
Examples of the connecting group

| | |
|---|---|
| 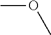 | 31 |
| 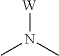 | 32 |
| 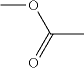 | 33 |
| 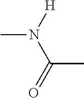 | 34 |
| 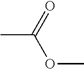 | 35 |
| 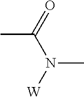 | 36 |
| 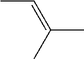 | 37 |
| 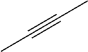 | 38 |
| 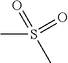 | 39 |
| 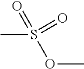 | 40 |
| 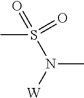 | 41 |

In some embodiments, the substitute R3 and/or R4 may be connected to the aromatic polycyclic conjugated molecule (Core) via at least one connecting group. The at least one connecting group may be selected from the list comprising CH2, CF2, SiR2O, CH2CH2O, wherein R is selected from the list comprising H, alkyl, and fluorine. In another embodiment of the composite organic compound, the one or more ionic groups include at least one ionic group selected from the list comprising [NR4]+, [PR4]+ as cation and [—CO2]-, [—SO3]-, [—SR5]-, [—PO3R]-, [—PR5]- as anion, wherein R is selected from the list comprising H, alkyl, and fluorine.

The Sharp polymers have hyperelectronic or ionic type polarizability. “Hyperelectronic polarization may be considered due to the pliant interaction of charge pairs of excitons, localized temporarily on long, highly polarizable molecules, with an external electric field [.] (Roger D. Hartman and Herbert A. Pohl, “Hyper-electronic Polarization in Macromolecular Solids”, Journal of Polymer Science: Part A-1 Vol. 6, pp. 1135-1152 (1968)).” Ionic type polarization can be achieved by limited mobility of ionic parts of the tethered/partially immobilized ionic liquid or zwitterion (Q). Additionally, other mechanisms of polarization such as dipole polarization and monomers and polymers possessing metal conductivity may be used independently or in combination with hyper-electronic and ionic polarization in aspects of the present disclosure.

In another aspect, the present disclosure provides a meta-dielectric, wherein a meta-dielectric is a dielectric that includes one or more Sharp polymers in the form of a composite organic compound characterized by polarizability and resistivity having the above general structural formula.

Further, characteristics of meta-dielectrics include a relative permittivity greater than or equal to 1,000 and resistivity greater than or equal to 1013 ohm/cm. Individually, the Sharp Polymers in a meta-dielectric may form column like supramolecular structures by pi-pi interaction. Said supramolecules of Sharp polymers allow formation of crystal structures of the meta-dielectric material. By way of using Sharp polymers in a dielectric material, polarization units are incorporated to provide the molecular material with high dielectric permeability. There are several mechanisms of polarization such as dipole polarization, ionic polarization, and hyper-electronic polarization of molecules, monomers and polymers possessing metal conductivity. All polarization units with the listed types of polarization may be used in aspects of the present disclosure. Further, Sharp polymers are composite materials which incorporate an envelope of insulating substituent groups that electrically isolate the supramolecules from each other in the dielectric crystal layer and provide high breakdown voltage of the energy storage molecular material. Said insulating substituent groups are resistive alkyl or fluro-alkyl chains covalently bonded to a polarizable core, forming the resistive envelope.

In order that the invention may be more readily understood, reference is made to the following examples, which are intended to be illustrative of the invention, but are not intended to be limiting the scope.

### EXAMPLE 1

This Example describes synthesis of one type of Sharp polymer according following structural scheme:

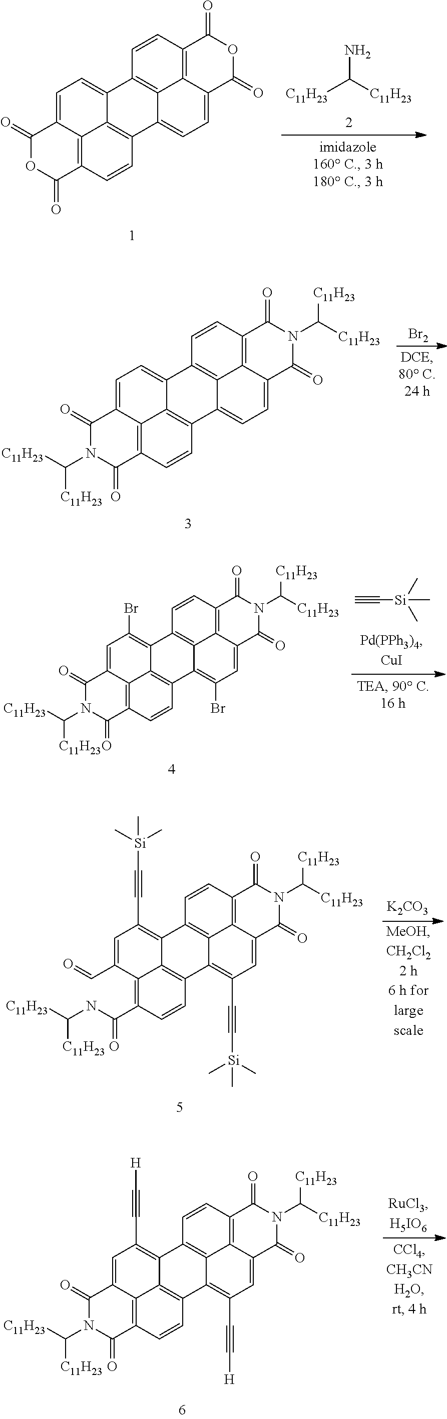

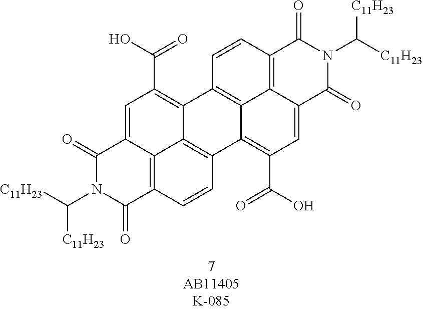

The process involved in the synthesis in this example may be understood in terms of the following five steps.

a) First Step:

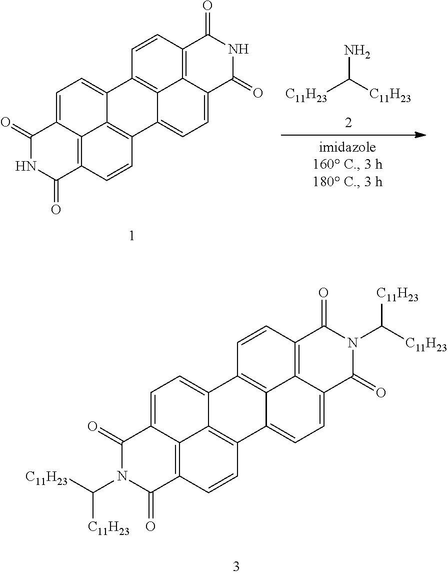

Anhydride 1 (60.0 g, 0.15 mol, 1.0 eq), amine 2 (114.4 g, 0.34 mol, 2.2 eq) and imidazole (686.0 g, 10.2 mol, 30 eq to 2) were mixed well into a 500 mL of round-bottom flask equipped with a bump-guarder. The mixture was degassed three times, stirred at 160° C. for 3 hr, 180° C. for 3hr, and cooled to rt. The reaction mixture was crushed into water (1000 mL) with stirring. Precipitate was collected with filtration, washed with water (2×500 mL), methanol (2×300 mL) and dried on high vacuum. The crude product was purified by flash chromatography column (CH₂Cl₂/hexane=1/1) to give 77.2 g (48.7%) of the desired product 3 as an orange solid. ¹H NMR (300 MHz, CDCl₃) δ 8.65-8.59 (m, 8H), 5.20-5.16 (m, 2H), 2.29-2.22 (m, 4H), 1.88-1.82 (m, 4H), 1.40-1.13 (m, 64H), 0.88-0.81 (t, 12H). Rf=0.68 (CH₂Cl₂/hexane=1/1).

b) Second Step:

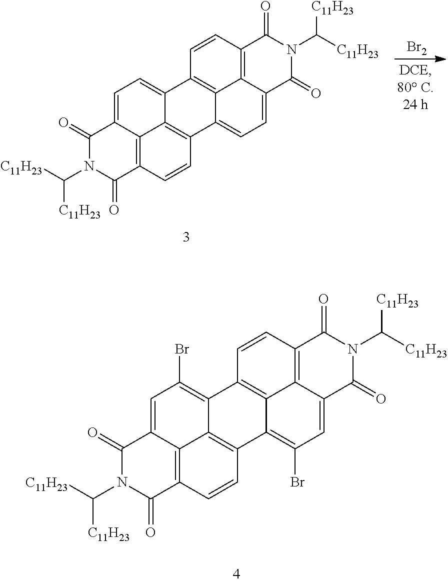

To a solution of the diimide 3 (30.0 g, 29.0 mmol, 1.0 eq) in dichloroethane (1500 mL) was added bromine (312.0 g, 1.95 mol, 67.3 eq). The resulting mixture was stirred at 80° C. for 36 hr, cooled, washed with 10% NaOH (aq, 2×1000 mL), water (100 ml), dried over Na₂SO₄, filtered and concentrated. The crude product was purified by flash chromatography column (CH₂Cl₂/hexanes=1/1) to give 34.0 g (98.2%) of the desired product 4 as a red solid. ¹H NMR (300 MHz, CDCl₃) δ 9.52 (d, 2H), 8.91 (bs, 2H), 8.68 (bs, 2H), 5.21-5.13 (m, 2H), 2.31-2.18 (m, 4H), 1.90-1.80 (m, 4H), 1.40-1.14 (m, 64H), 0.88-0.81 (t, 12H). Rf=0.52 (CH₂Cl₂/hexanes=1/1).

c) Third Step

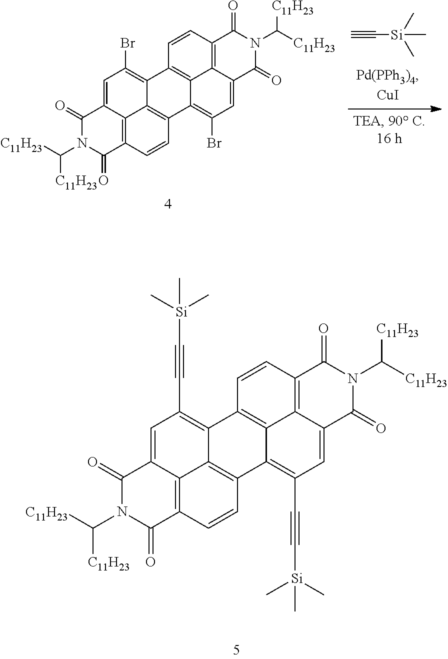

To a solution of the di-bromide 4 (2.0 g, 1.68 mmol, 1.0 eq) in triethylamine (84.0 mL) was added CuI (9.0 mg, 0.048 mmol, 2.8 mol %) and (trimethylsilyl)acetylene (80.49 g, 5.0 mmol, 3.0 eq). The mixture was degassed three times. Catalyst Pd(PPh₃)₄ (98.0 mg, 0.085 mmol, 5.0 mol %) was added. The mixture was degassed three times, stirred at 90° C. for 24 hr, cooled, passed through a pad of Celite, and concentrated. The crude product was purified by flash chromatography column (CH₂Cl₂/hexane=1/1) to give 1.8 g (87.2%) of the desired product 5 as a dark-red solid. ¹H NMR (300 MHz, CDCl₃) δ 10.24-10.19 (m, 2H), 8.81 (bs, 2H), 8.65 (bs, 2H), 5.20-5.16 (m, 2H), 2.31-2.23 (m, 4H), 1.90-1.78 (m, 4H), 1.40-1.15 (m, 72H), 0.84-0.81 (t, 12H), 0.40 (s, 18H). Rf=0.72 (CH₂Cl₂/hexane=1/1).

d) Fourth Step

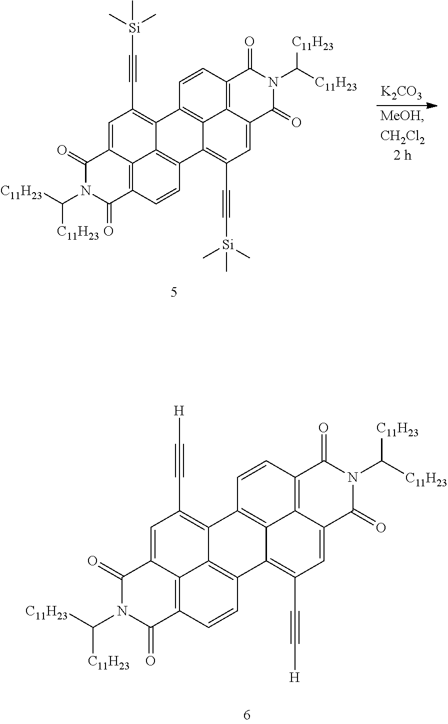

To a solution of diimide 5 (1.8 g, 1.5 mmol, 1.0 eq) in a mixture of MeOH/DCM (40.0 mL/40.0 mL) was added K₂CO₃ (0.81 g, 6.0 mmol, 4.0 eq). The mixture was stirred at room temperature for 1.5 hr, diluted with DCM (40.0 mL), washed with water, brine, dried over Na₂SO₄, filtered and concentrated. The crude product was purified by flash chromatography column (CH₂Cl₂) to give 1.4 g (86.1%) of the desired product 6 as a dark-red solid. ¹H NMR (300 MHz, CDCl₃) δ 10.04-10.00 (m, 2H), 8.88-8.78 (m, 2H), 8.72-8.60 (m, 2H), 5.19-5.14 (m, 2H), 3.82-3.80 (m, 2H), 2.31-2.23 (m, 4H), 1.90-1.78 (m, 4H), 1.40-1.05 (m, 72H), 0.85-0.41 (t, 12H). Rf=0.62 (CH₂Cl₂).

e) Fifth Step

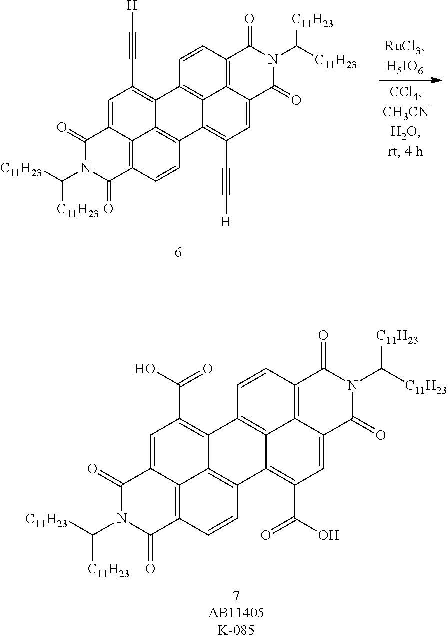

To a suspension of alkyne 6 (1.4 g, 1.3 mmol, 1.0 eq) in a mixture of CCl₄/CH₃CN/H₂O (6 mL/6 mL/12 mL) was added periodic acid (2.94 g, 12.9 mmol, 10.0 eq) and RuCl₃ (28.0 mg, 0.13 mmol, 10 mol %). The mixture was stirred at room temperature under nitrogen for 4 hours, diluted with DCM (50 mL), washed with water, brine, dried over Na₂SO₄, filtered and concentrated. The crude product was purified by flash chromatography column (10% MeOH/CH₂Cl₂) to give 1.0 g (68.5%) of the desired product 7 as a dark-red solid. ¹H NMR (300 MHz, CDCl₃) d 8.90-8.40 (m, 6H), 5.17-5.00 (m, 2H), 2.22-2.10 (m, 4H), 1.84-1.60 (m, 4H), 1.41-0.90 (m, 72H), 0.86-0.65 (t, 12H). Rf=0.51 (10% MeOH/CH₂Cl₂).

### EXAMPLE 2

This Example describes synthesis of a Sharp polymer according following structural scheme:

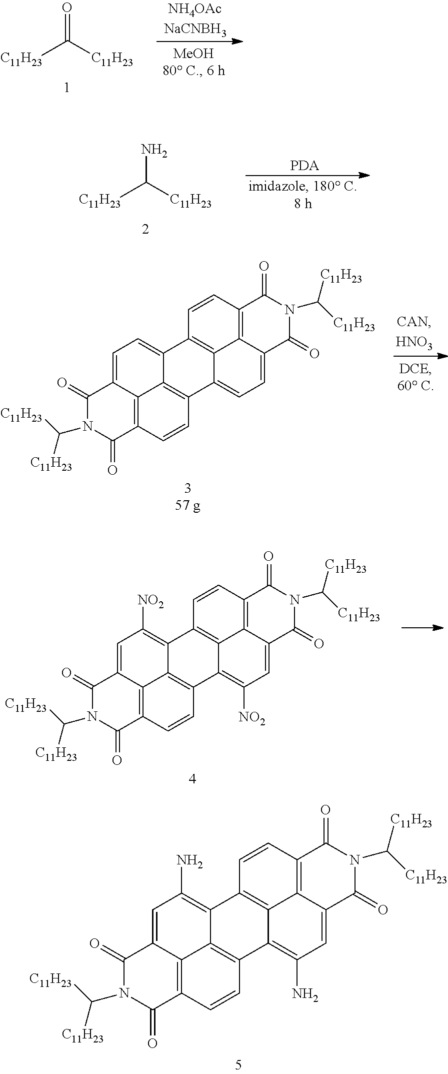

The process involved in the synthesis in this example may be understood in terms of the following four steps.

a) First Step:

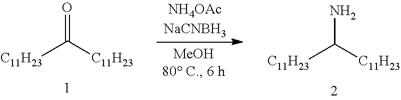

To a solution of the ketone 1 (37.0 g, 0.11 mol, 1.0 eq) in methanol (400 mL) was added ammonium acetate (85.3 g, 1.11 mol, 10.0 eq) and NaCNBH3 (28.5 g, 0.44 mol, 4.0 eq) in portions. The mixture was stirred at reflux for 6 hours, cooled to room temperature and concentrated. Sat. NaHCO3 (500 mL) was added to the residue and the mixture was stirred at room temperature for 1 hour. Precipitate was collected by filtration, washed with water (4×100 mL), dried on a high vacuum to give 33.6 g (87%) of the amine 2 as a white solid.

b) Second Step:

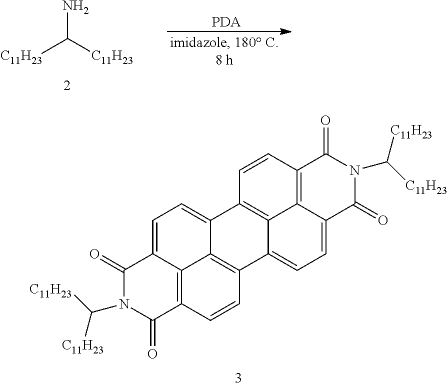

Mixed well the amine 2 (20.0 g, 58.7 mmol, 2.2 equ), 3,4,9,10-perylenetetracarboxylic dianhydride (10.5 g, 26.7 mmol, 1.0 eq) and imidazole (54.6 g, 0.80 mmol, 30 eq to diamine) into a 250 mL round-bottom flask equipped with a rotavap bump guard. The mixture was degassed (vacuum and fill with N2) three times and stirred at 160 oC for 6 hrs. After cooling to rt, the reaction mixture was crushed into water (700 mL), stirred for 1 hr, and filtered through a filter paper to collected precipitate which was washed with water (3×300 mL) and methanol (3×300 mL), dried on a high vacuum to give 23.1 g (83.5%) of the diamidine 3 as a orange solid. Pure diamidine 3 (20.6 g) was obtained by flash chromatography column (DCM/hexanes=1/1).

c) Third Step:

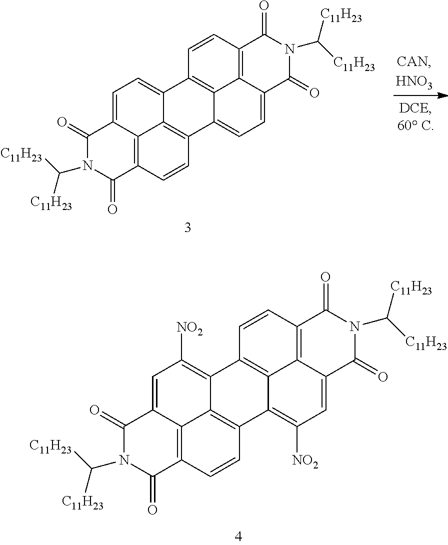

To DCE (2.0 L) was added compound 3 (52.0 g, 50.2 mmol, 1.0 eq), acetic acid (500 mL) and fuming nitric acid (351.0 g, 5.0 mol, 100.0 eq) with caution. To the mixture was added ammonium cerium(IV) nitrate (137.0 g, 0.25 mol, 5.0 eq). The reaction was stirred at 60 oC for 48 hrs. After cooling to rt, the reaction mixture was crushed into water (1.0 L). The organic phase was washed with water (2×1.0 L), saturated NaHCO3 solution (1×1.0 L) and brine (1×1.0 L), dried over sodium sulfate, filtered and concentrated. The residue was purified with column chromatography to give 46.7 g (82%) of compound 4 as a dark red solid. 1H NMR (300 MHz, CDCl3) δ 0.84 (t, 12H), 1.26 (m, 72H), 1.83 (m, 4H), 2.21 (m, 4H), 5.19 (m, 2H), 8.30 (m, 2H), 8.60-8.89 (m, 4H).

d) Fourth Step:

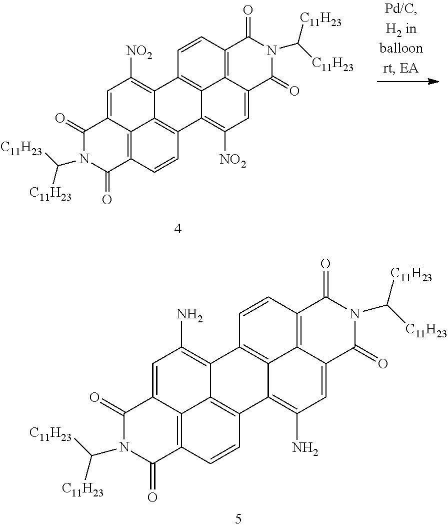

A mixture of compound 4 (25 g, 22.2 mmol, 1.0 eq) and Pd/C (2.5 g, 0.1 eq) in EtOAc (125.0 mL) was stirred at room temperature for 1 hour. The solid was filtered off (Celite) and washed with EtOAc (5 mL×2). The filtrate was concentrated to afford the compound 5 (23.3 g, 99%) as a dark blue solid. 1H NMR (300 MHz, CDCl3) δ 0.84 (t, 12H), 1.24 (m, 72H), 1.85 (m, 4H), 2.30 (m, 4H), 5.00 (s, 2H), 5.10 (s, 2H), 5.20 (m, 2H), 7.91-8.19 (dd, 2H), 8.40-8.69 (dd, 2H), 8.77-8.91 (dd, 2H).

Furuta co-polymers and para-Furuta polymers (herein referred to collectively as Furuta Polymers unless otherwise specified) are polymeric compounds with insulating tails, and linked/tethered/partially immobilized polarizable ionic groups. The insulating tails are hydrocarbon (saturated and/or unstaturate), fluorocarbon, siloxene, and/or polyethylene glycol linear or branched chains covalently bonded to the co-polymer backbone. The tails act to insulate the polarizable tethered/partially immobilized ionic molecular components and ionic pairs from other ionic groups and ionic group pairs on the same or parallel co-polymers, which favorably allows discrete polarization of counter ionic liquid pairs or counter Q groups (i.e. polarization of cationic liquid and anionic anionic liquid tethered/partially immobilized to parallel Furuta polymers) with limited or no interaction of ionic fields or polarization moments of other counter ionic group pairs partially immobilized on the same or parallel co-polymer chains. Further, the insulating tails electrically insulate supra-structures of Furuta polymers from each other. Parallel Furuta polymers may arrange or be arranged such that counter ionic groups (i.e. tethered/partially immobilized ionic groups (Qs) of cation and anion types (sometimes known as cationic Furuta polymers and anionic Furuta polymers)) are aligned opposite from one another.

A Furuta co-polymer has the following general structural formula:

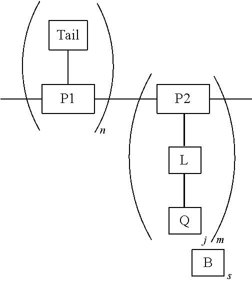

wherein backbone structure of the co-polymer comprises structural units of first type P1 and structural units of second type P2 both of which randomly repeat and are independently selected from the list comprising acrylic acid, methacrylate, repeat units of polypropylene (—[CH₂—CH(CH₃)]—), repeat units of polyethylene (—[CH₂]—), siloxane, or repeat units of polyethylene terephthalate (sometimes written poly(ethylene terephthalate)) for which the repeat unit may be expressed as —CH₂—CH₂—O—CO—C₆H₄—CO—O—. Parameter n is the number of the P1 structural units in the backbone structure which is in the range from 3 to 100,000 and m is number of the P2 structural units in the backbone structure which is in the range from 3 to 100,000. Further, the first type structural unit (P1) has a resistive substitute Tail which is oligomers of polymeric material with HOMO-LUMO gap no less than 2 eV. Additionally, the second type of structural units (P2) has an ionic functional group Q which is connected to P2 via a linker group L. The parameter j is a number of functional groups Q attached to the linker group L, which may range from 0 to 5. Wherein the ionic functional group Q comprises one or more ionic liquid ions (from the class of ionic compounds that are used in ionic liquids), zwitterions, or polymeric acids. Further, an energy interaction of the ionic Q groups may be less than kT, where k is Boltzmann constant and T is the temperature of environment. Still further, parameter B is a counter ion which is a molecule or molecules or oligomers that can supply the opposite charge to balance the charge of the co-polymer. Wherein, s is the number of the counter ions.

The present disclosure includes implementations in which the meta-dielectric includes an organic co-polymeric compound having the structure described above. In one embodiment of the organic co-polymeric compound, the resistive substitute Tails are independently selected from the list comprising oligomers of polypropylene (PP), oligomers of polyethylene terephthalate (PET), oligomers of polyphenylene sulfide (PPS), oligomers of polyethylene naphthalate (PEN), oligomers of polycarbonate (PP), polystyrene (PS), and oligomers of polytetrafluoroethylene (PTFE). In another embodiment of the organic co-polymeric compound, the resistive substitutes Tail are independently selected from alkyl, aryl, substituted alkyl, substituted aryl, fluorinated alkyl, chlorinated alkyl, branched and complex alkyl, branched and complex fluorinated alkyl, branched and complex chlorinated alkyl groups, and any combination thereof, and wherein the alkyl group is selected from methyl, ethyl, propyl, butyl, iso-butyl and tert-butyl groups, and the aryl group is selected from phenyl, benzyl and naphthyl groups. The resistive substitute Tail may be added after polymerization.

In yet another aspect of the present disclosure, it is preferable that the HOMO-LUMO gap is no less than 4 eV. In still another aspect of the present disclosure, it is even more preferable that the HOMO-LUMO gap is no less than 5 eV. The ionic functional group Q comprises one or more ionic liquid ions from the class of ionic compounds that are used in ionic liquids, zwitterions, or polymeric acids. The energy of interaction between Q group ions on discrete P2 structural units may be less than kT, where k is Boltzmann constant and T is the temperature of environment. The temperature of environment may be in range between −60 C of and 150 C. The preferable range of temperatures is between −40 C and 100 C. Energy interaction of the ions depends on the effective radius of ions. Therefore, by increasing the steric hindrance between ions it is possible to reduce energy of interaction of ions. In one embodiment of the present invention, at least one ionic liquid ion is selected from the list comprising [NR4]+, [PR4]+ as cation and [—CO2]-, [—SO3]-, [—SR5]-, [—PO3R]-, [—PR5]- as anion, wherein R is selected from the list comprising H, alkyl, and fluorine. The functional group Q may be charged after or before polymerization. In another embodiment of the present invention, the linker group L is oligomer selected from structures 42 to 47 as given in Table 3.

TABLE 3
Examples of the oligomer linker group

| | |
|---|---|
| 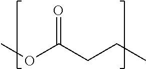 | 42 |
| 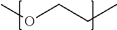 | 43 |
| 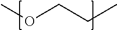 | 44 |
| 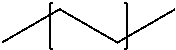 | 45 |
| 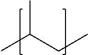 | 46 |
| 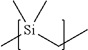 | 47 |

In yet another embodiment of the present invention, the linker group L is selected from structures 48 to 56 as given in Table 4.

TABLE 4
Examples of the linker group

| | |
|---|---|
| 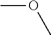 | 48 |
| 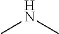 | 49 |
| 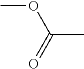 | 50 |
| 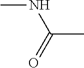 | 51 |
| 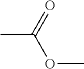 | 52 |
| 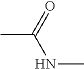 | 53 |
| 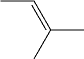 | 54 |
| 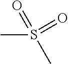 | 55 |
| 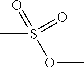 | 56 |

In yet another embodiment of the present invention, the linker group L may be selected from the list comprising CH₂, CF₂, SiR₂O, and CH2CH2O, wherein R is selected from the list comprising H, alkyl, and fluorine. The ionic functional group Q and the linker groups L may be added after polymerization.

In another aspect, the present disclosure provides a dielectric material (sometimes called a meta-dielectric) comprising of one or more of the class of Furuta polymers comprising protected or hindered ions of zwitterion, cation, anion, or polymeric acid types described hereinabove. The meta-dielectric material may be a mixture of zwitterion type Furuta polymers, or positively charged (cation) Furuta polymers and negatively charged (anion) Furuta polymers, polymeric acid Furuta polymers, or any combination thereof. The mixture of Furuta polymers may form or be induced to form supra-structures via hydrophobic and ionic interactions. By way of example, but not limiting in scope, the cation on a positively charged Furuta polymer replaces the B counter ions of the anion on a negatively charged Furuta polymer parallel to the positively charged Furuta polymer and vice versa; and the resistive Tails of neighboring Furuta polymers further encourages stacking via van der Waals forces, which increases ionic group isolation. Meta-dielectrics comprising both cationic and anionic Furuta polymers have a 1:1 ratio of cationic and anionic Furuta polymers.

The Tails of hydrocarbon (saturated and/or unsaturated), fluorocarbon, siloxane, and/or polyethylene glycol linear or branched act to insulate linked/tethered/partially immobilized polarizable ionic liquids, zwitterions, or polymeric acids (ionic Q groups). The Tails insulate the ionic Q groups from other ionic Q groups on the same or parallel Furuta polymer via steric hindrance of the ionic Q groups' energy of interaction, which favorably allows discrete polarization of the ionic Q groups (i.e. polarization of cationic liquid and anionic liquid tethered/partially immobilized to parallel Furuta polymers). Further, the Tails insulate the ionic groups of supra-structures from each other. Parallel Furuta polymers may arrange or be arranged such that counter ionic liquids (i.e. tethered/partially immobilized ionic liquids (Qs) of cation and anion types) are aligned opposite from one another (sometimes known as cationic Furuta polymers and anionic Furuta polymers).

The Furuta polymers have hyperelectronic or ionic type polarizability. “Hyperelectronic polarization may be considered due to the pliant interaction of charge pairs of excitons, localized temporarily on long, highly polarizable molecules, with an external electric field [.] (Roger D. Hartman and Herbert A. Pohl, “Hyper-electronic Polarization in Macromolecular Solids”, Journal of Polymer Science: Part A-1 Vol. 6, pp. 1135-1152 (1968)).” Ionic type polarization can be achieved by limited mobility of ionic parts of the tethered/partially immobilized ionic liquid or zwitterion (Q). Additionally, other mechanisms of polarization such as dipole polarization and monomers and polymers possessing metal conductivity may be used independently or in combination with hyper-electronic and ionic polarization in aspects of the present disclosure.

Further, a meta-dielectric layer may be comprised of one or more types of zwitterion Furuta polymer and/or selected from the anionic Q⁺ group types and cationic Q⁻ group types and/or polymeric acids, having the general configuration of Furuta polymers:

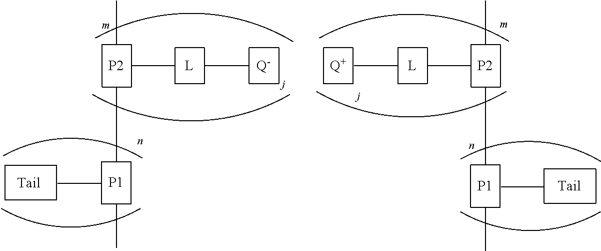

In order that the invention may be more readily understood, reference is made to the following examples of synthesis of Furuta co-polymers, which are intended to be illustrative of the invention, but are not intended to be limiting the scope.

### EXAMPLE 3

Carboxylic acid co-polymer P002. To a solution of 1.02 g (11.81 mmol) of methacrylic acid and 4.00 g (11.81 mmol) of stearylmethacrylate in 2.0 g isopropanol was added a solution of 0.030 g 2,2′-azobis(2-methylpropionitrile) (AIBN) in 5.0 g of toluene. The resulting solution was heated to 80 C for 20 hours in a sealed vial, after which it became noticeably viscous. NMR shows <2% remaining monomer. The solution was used without further purification in film formulations and other mixtures.

### EXAMPLE 4

Amine co-polymer P011. To a solution of 2.52 g (11.79 mmol) of 2-(diisopropylamino)ethyl methacrylate and 3.00 g (11.79 mmol) of laurylmethacrylate in 2.0 g toluene was added a solution of 0.030 g 2,2′-azobis(2-methylpropionitrile) (AIBN) in 4.0 g of toluene. The resulting solution was heated to 80 C for 20 hours in a sealed vial, after which it became noticeably viscous. NMR shows <2%remaining monomer. The solution was used without further purification in film formulations and other mixtures.

### EXAMPLE 5

Carboxylic acid co-polymer and amine co-polymer mixture. 1.50 g of a 42 wt % by solids solution of P002 was added to 1.24 g of a 56 wt % solution of P011 with 1 g of isopropanol and mixed at 40 C for 30 minutes. The solution was used without further purification.

A para-Furuta polymer has repeat units of the following general structural formula:

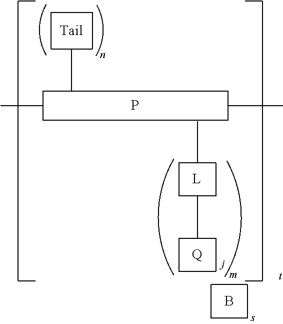

wherein a structural unit P comprises a backbone of the copolymer, which is independently selected from the list comprising acrylic acid, methacrylate, repeat units for polypropylene (PP) (—[CH₂—CH(CH₃)]—), repeat units for polyethylene (PE) (—[CH₂]—), siloxane, or repeat units of polyethylene terephthalate (sometimes written poly(ethylene terephthalate)) for which the repeat unit may be expressed as —CH₂—CH₂—O—CO—C₆H₄—CO—O—. Wherein the first type of repeat unit (Tail) is a resistive substitute in the form of an oligomer of a polymeric material. The resistive substitute preferably has a HOMO-LUMO gap no less than 2 eV. The parameter n is a number of Tail repeat units on the backbone P structural unit, and is in the range from 3 to 100,000. Further, the second type of repeat units (-L-Q) include an ionic functional group Q which is connected to the structural backbone unit (P) via a linker group L, and m is number of the -L-Q repeat units in the backbone structure which is in the range from 3 to 100,000. Additionally, the ionic functional group Q comprises one or more ionic liquid ions (from the class of ionic compounds that are used in ionic liquids), zwitterions, or polymeric acids. An energy of interaction of the ionic Q groups may be less than kT, where k is Boltzmann constant and T is the temperature of environment. Still further, the parameter t is average of para-Furuta polymer repeat units, ranging from 6 to 200,000. Wherein B's are counter ions which are molecules or oligomers that can supply the opposite charge to balance the charge of the co-polymer, s is the number of the counter ions.

The present disclosure provides an organic polymeric compound. In one embodiment of the organic polymeric compound, the resistive substitute Tails are independently selected from the list comprising polypropylene (PP), polyethylene terephthalate (PET), polyphenylene sulfide (PPS), polyethylene naphthalate (PEN), polycarbonate (PP), polystyrene (PS), and polytetrafluoroethylene (PTFE). In another embodiment of the organic polymeric compound, the resistive substitutes Tail are independently selected from alkyl, aryl, substituted alkyl, substituted aryl, fluorinated alkyl, chlorinated alkyl, branched and complex alkyl, branched and complex fluorinated alkyl, branched and complex chlorinated alkyl groups, and any combination thereof, and wherein the alkyl group is selected from methyl, ethyl, propyl, butyl, iso-butyl and tert-butyl groups, and the aryl group is selected from phenyl, benzyl and naphthyl groups. The resistive substitute Tail may be added after polymerization. In yet another embodiment of the present disclosure, it is preferable that the HOMO-LUMO gap is no less than 4 eV. In still another embodiment of the present disclosure, it is even more preferable that the HOMO-LUMO gap is no less than 5 eV. The ionic functional group Q comprises one or more ionic liquid ions from the class of ionic compounds that are used in ionic liquids, zwitterions, or polymeric acids. Energy of interaction between Q group ions on discrete P structural units may be less than kT, where k is Boltzmann constant and T is the temperature of environment. The temperature of environment may be in range between −60 C of and 150 C. The preferable range of temperatures is between −40 C and 100 C. Energy interaction of the ions depends on the effective radius of ions. Therefore, by increasing the steric hindrance between ions it is possible to reduce energy of interaction of ions. In one embodiment of the present invention, at least one ionic liquid ion is selected from the list comprising [NR₄]⁺, [PR₄]⁺ as cation and [—CO₂]⁻, [—SO₃]⁻, [—SR_{S}]⁻, [—PO₃R]⁻, [—PR_{S}]⁻ as anion, wherein R is selected from the list comprising H, alkyl, and fluorine. The functional group Q may be charged after or before polymerization. In another embodiment of the present invention, the linker group L is oligomer selected from structures 42 to 47 as given in Table 3 or structures 48 to 56 in Table 4.

In yet another embodiment of the present invention, the linker group L is selected from the list comprising CH₂, CF₂, SiR₂O, and CH2CH2O, wherein R is selected from the list comprising H, alkyl, and fluorine. The ionic functional group Q and the linker groups L may be added after polymerization.

In another aspect, the present disclosure provides a dielectric material (sometimes called a meta-dielectric) comprising of one or more of the class of para-Furuta polymers comprising protected or hindered ions of zwitterion, cationic liquid ions, anionic liquid ions, or polymeric acid types described hereinabove. The meta-dielectric material may be a mixture of zwitterion type para-Furuta polymers, or positively charged (cation) para-Furuta polymers and negatively charged (anion) para-Furuta polymers, polymeric acid para-Furuta polymers, or any combination thereof. The mixture of para-Furuta polymers may form or be induced to form supra-structures via hydrophobic and ionic interactions. By way of example, but not limiting in scope, the cation(s) on a positively charged para-Furuta polymer replaces the B counter ions of the anion(s) on a negatively charged para-Furuta polymer parallel to the positively charged para-Furuta polymer and vice versa; and the resistive Tails of neighboring para-Furuta polymers further encourages stacking via van der Waals forces, which increases ionic group isolation. Meta-dielectrics comprising both cationic and anionic para-Furuta polymers preferably have a 1:1 ratio of cationic and anionic para-Furuta polymers.

The Tails of hydrocarbon (saturated and/or unsaturated), fluorocarbon, siloxane, and/or polyethylene glycol linear or branched act to insulate linked/tethered/partially immobilized polarizable ionic liquids, zwitterions, or polymeric acids (ionic Q groups). The Tails insulate the ionic Q groups from other ionic Q groups on the same or parallel para-Furuta polymer via steric hindrance of the ionic Q groups' energy of interaction, which favorably allows discrete polarization of the ionic Q groups (i.e. polarization of cationic liquid and anionic liquid tethered/partially immobilized to parallel para-Furuta polymers). Further, the Tails insulate the ionic groups of supra-structures from each other. Parallel para-Furuta polymers may arrange or be arranged such that counter ionic liquids (i.e. tethered/partially immobilized ionic liquids (Qs) of cation and anion types) are aligned opposite from one another (sometimes known as cationic para-Furuta polymers and anionic para-Furuta polymers).

The para-Furuta polymers have hyperelectronic or ionic type polarizability. “Hyperelectronic polarization may be considered due to the pliant interaction of charge pairs of excitons, localized temporarily on long, highly polarizable molecules, with an external electric field [.] (Roger D. Hartman and Herbert A. Pohl, “Hyper-electronic Polarization in Macromolecular Solids”, Journal of Polymer Science: Part A-1 Vol. 6, pp. 1135-1152 (1968)).” Ionic type polarization can be achieved by limited mobility of ionic parts of the tethered/partially immobilized ionic liquid or zwitterion (Q). Additionally, other mechanisms of polarization such as dipole polarization and monomers and polymers possessing metal conductivity may be used independently or in combination with hyper-electronic and ionic polarization in aspects of the present disclosure.

Further, a meta-dielectric layer may be comprised of one or more types of zwitterion para-Furuta polymer and/or selected from the anionic Q group types and cationic Q group types and/or polymeric acids, which may have the following general arrangement of para-Furuta polymers:

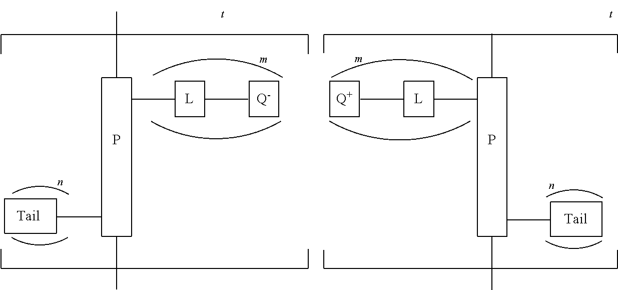

Other meta-dielectric materials that could be used include oligomers composed of repeating peryline derivative structures with various substituents and polymers bearing conjugated azo-aromatic pendant groups. Such compounds are described in U.S. patent application Ser. No. 15/090,509 (Attorney Docket No. CSI-051), Ser. No. 15/043,247 (Attorney Docket No. CSI-051B), Ser. No. 15/194,224 (Attorney Docket No. CSI-044), Ser. No. 14/919,337 (Attorney Docket No. CSI-022), and 62/318,134 (Attorney Docket No. CSI-050) which are hereby incorporated in their entirety by reference.

A meta-dielectric is defined here as a dielectric material comprised of one or more types of structured polymeric materials (SPMs) having a relative permittivity greater than or equal to 1000 and resistivity greater than or equal to 10¹⁶ ohm·cm. Individually, the SPMs in a meta-dielectric may form column like supramolecular structures by pi-pi interaction or hydrophilic and hydrophobic interactions. Said supramolecules of SPMs may permit formation of crystal structures of the meta-dielectric material. By way of using SPMs in a dielectric material, polarization units are incorporated to provide the molecular material with high dielectric permeability. There are several mechanisms of polarization such as dipole polarization, ionic polarization, and hyper-electronic polarization of molecules, monomers and polymers possessing metal conductivity. All polarization units with the listed types of polarization may be used in aspects of the present disclosure. Further, SPMs are composite materials which incorporate an envelope of insulating substituent groups that electrically isolate the supramolecules from each other in the dielectric layer and provide high breakdown voltage of the energy storage molecular material. Said insulating substituent groups are hydrocarbon (saturated and/or unsaturated), fluorocarbon, siloxane, and/or polyethylene glycol linear or branched chains covalently bonded to a polarizable core or co-polymer backbone, forming the resistive envelope.

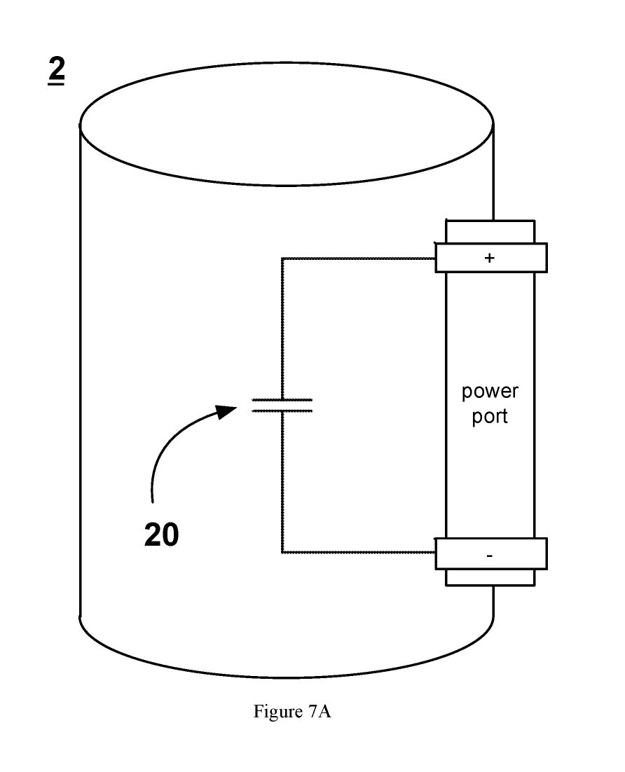

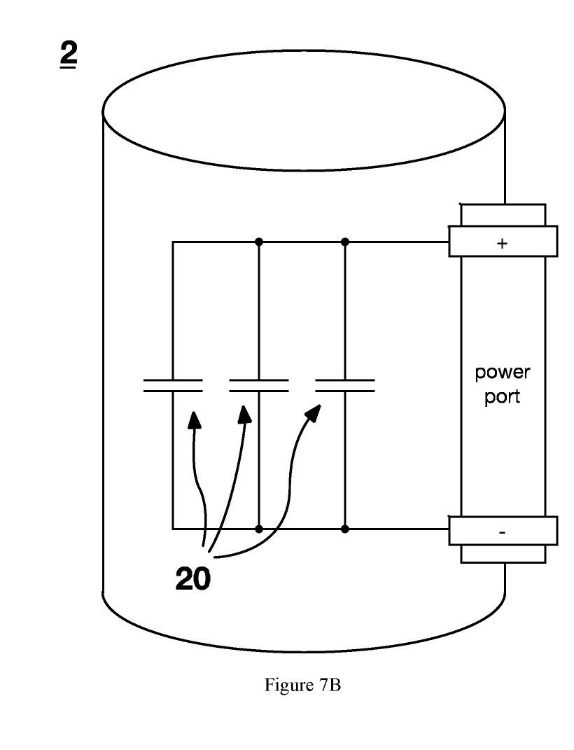

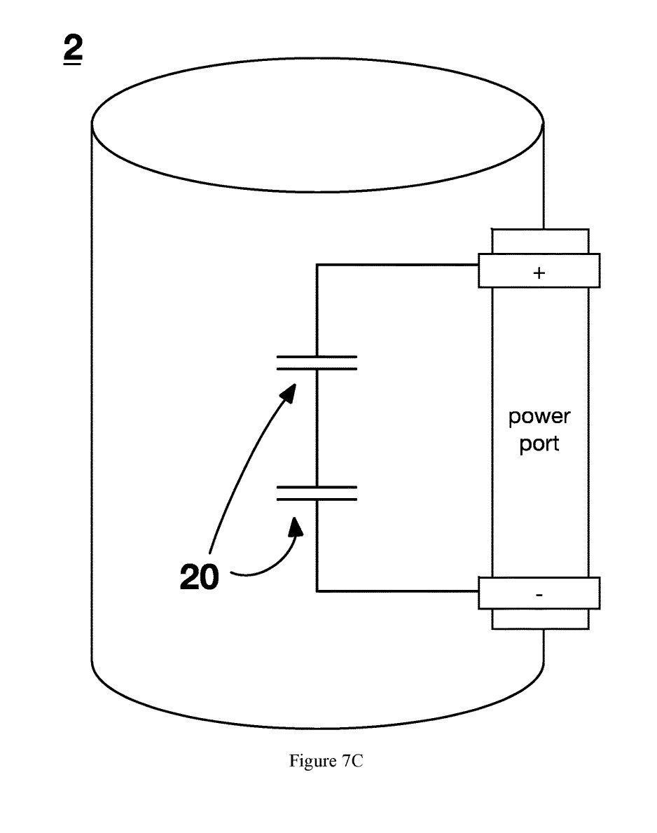

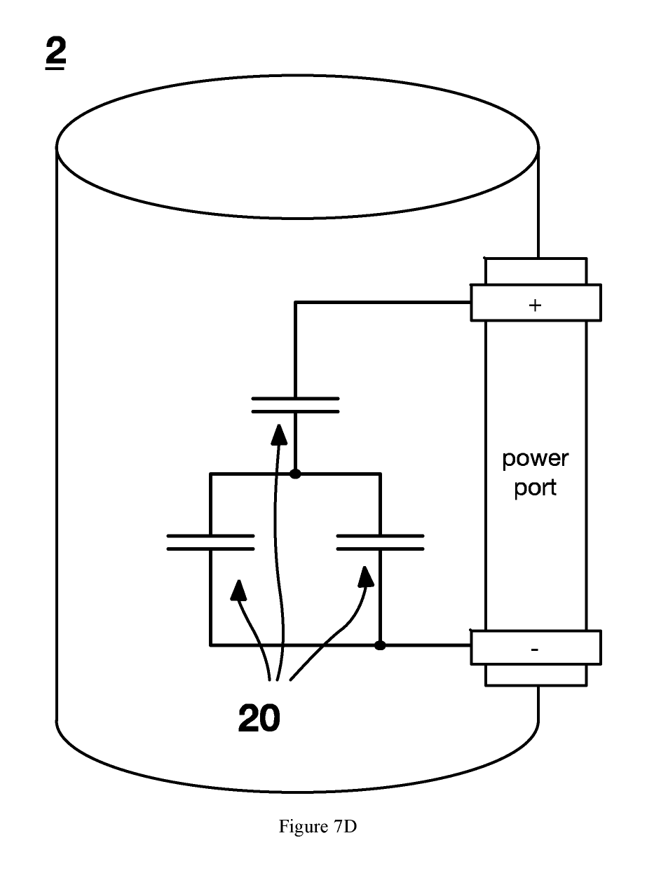

As depicted in FIG. 10, in one embodiment of the energy storage cell 1, each of the one or more meta-capacitors 20 comprises a first electrode 21, a second electrode 22, and a meta-dielectric material layer 23 disposed between said first and second electrodes. The electrodes 21, 22 may be made of a metal, such as copper, zinc, or aluminum or other conductive material and are generally planar in shape. In one implementation, the electrodes and meta-dielectric material layer 23 are in the form of long strips of material that are sandwiched together and wound into a coil along with an insulating material, e.g., a plastic film such as polypropylene or polyester to prevent electrical shorting between the electrodes 21, 22. Examples of such coiled capacitor energy storage devices are described in detail in commonly-assigned U.S. patent application Ser. No. 14/752,600, filed Jun. 26, 2015, the entire contents of which are incorporated herein by reference. Although a single meta-capacitor 20 is shown for convenience in FIG. 10, aspects of the present disclosure are not limited to such implementations. As illustrated in FIGS. 7A, 7B, 7C, 7D, those skilled in the art will recognize that the capacitive energy storage device 2 may include multiple meta-capacitors 20 connected in parallel, as in FIG. 7B, to provide a desired amount of energy storage capacity that scales roughly with the number of meta-capacitors in parallel. Alternatively, the capacitive energy storage device 2 may include two or more meta-capacitors connected in series to accommodate a desired voltage level, as in FIG. 7C. In addition, the capacitive energy storage device 2 may include combinations of three or more meta-capacitors in a capacitor network involving various series and parallel combinations, as in FIG. 7D. For example, there may be three capacitor combinations connected in parallel with each other with each combination having two capacitors connected in series.

The meta-dielectric material 23 may be characterized by a dielectric constant κ greater than about 100 and a breakdown field E_{bd} greater than or equal to about 0.01 volts (V)/nanometer (nm). The dielectric constant κ may be greater than or equal to about 100, 200, 300, 400, 500, 1000, 2000, 3000, 4000, 5000, 6000, 7000, 8000, 9000, 10,000, or 100,000. The breakdown field may be greater than about 0.01 V/nm, 0.05 V/nm, 0.1 V/nm, 0.2 V/nm, 0.3 V/nm, 0.4 V/nm, 0.5 V/nm, 1 V/nm, or 10 V/nm. By way of example, and not by way of limitation, the meta-dielectric material 23 may be characterized by a dielectric constant κ between about 100 and about 1,000,000 and a breakdown field Ebd between about 0.01 V/nm and about 2.0 V/nm.

In yet another implementation, the capacitive energy storage devices may comprise more than one of the meta-capacitors connected in series or parallel. In still another implementation, the capacitive energy storage device may further comprise a cooling mechanism 30. In some implementations, the cooling can be passive, e.g., using radiative cooling fins on the capacitive energy storage device 2 and DC-voltage conversion device 3. Alternatively, a fluid such as air, water or ethylene glycol can be used as a coolant in an active cooling system. By way of example, and not by way of limitation, the cooling system 30 may include conduits in thermal contact with the capacitive energy storage device 2 and DC-voltage conversion device 3. The conduits are filled with a heat exchange medium, which may be a solid, liquid or gas. In some implementations, the cooling mechanism may include a heat exchanger configured to extract heat from the heat exchange medium. In other implementations, the cooling mechanism 30 may include conduits in the form of cooling fins on the capacitive energy storage device 2 and DC-voltage conversion device 3 and the heat exchange medium is air that is blown over the cooling fins, e.g., by a fan. In another embodiment of the present invention, the heat exchanger 32 may include a phase-change heat pipe configured to carry out cooling. The cooling carried out by the phase-change heat pipe may involve a solid to liquid phase change (e.g., using melting of ice or other solid) or liquid to gas phase change (e.g., by evaporation of water or alcohol) of a phase change material. In yet another implementation, the conduits or heat exchanger 32 may include a reservoir containing a solid to liquid phase change material, such as paraffin wax.

Referring again to FIGS. 10, 11 and 12 the DC-voltage conversion device 3 may include a buck converter for applications in which Vₒᵤₜ<Vᵢₙ, a boost converter for applications in which Vₒᵤₜ>Vᵢₙ, or a bidirectional buck/boost converter for applications in which Vₒᵤₜ<Vᵢₙ in certain situations and Vₒᵤₜ>Vᵢₙ in other situations.

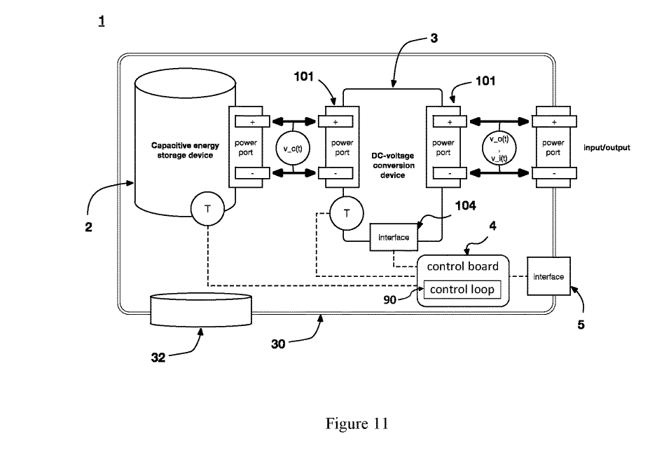

In still another embodiment of the energy storage cell (see, FIG. 11) the DC-voltage conversion device 3 may be connected to a control board 4 containing suitable logic circuitry, e.g., microprocessor, microcontroller, application specific integrated circuit (ASIC), field programmable gate array (FPGA), a complex programmable logic device (CPLD), capable of implementing closed loop control processes 90 and (optionally) a communication interface 5, as well as an analog to digital converter coupled to sensors on the DC-voltage conversion device 3, e.g., voltage sensors V for the input voltage Vᵢₙ and the output voltage Vₒᵤₜ, current sensors A for current I_{sd} to/from the capacitive energy storage device 2 and/or current I_{vc} to/from the DC-voltage conversion device 3, temperature sensors T on the capacitive energy storage device and/or DC-voltage conversion device. In some implementations, the control board 4 may be integrated into the DC-voltage conversion device 3. The conversion device 3 may contain a buck regulator, a boost regulator, buck and boost regulators with separate input/outputs, a bi-directional boost/buck regulator, or a split-pi converter and the control board 4 may be configured to maintain a constant output voltage Vₒᵤₜ from the DC-voltage conversion device during discharge, and/or charge the capacitor at a more-or-less constant current while maintaining a stable input voltage.

By way of example, and not by way of limitation, the control board 4 may be based on a controller for a bidirectional buck/boost converter. In such a configuration, the control board 4 stabilizes the output voltage of the DC-voltage conversion device according to the following algorithm forming the control loop 90:
a) determining a target output voltage level for the energy storage system,
b) measuring the voltage of a capacitive energy storage device,
c) configuring a bidirectional buck/boost converter to buck down the voltage and direct current in the output direction IF the voltage on the capacitive energy storage device is higher than the desired output voltage and the desired outcome is to discharge the device,
d) configuring a bidirectional buck/boost converter to boost up the voltage and direct current in the output direction IF the voltage on the capacitive energy storage device is lower than the desired output voltage and the desired outcome is to discharge the device,
e) configuring a bidirectional buck/boost converter to buck down the voltage and direct current in the input direction IF the voltage on the capacitive energy storage device is lower than the desired input voltage and the desired outcome is to charge the device,
f) configuring a bidirectional buck/boost converter to boost up the voltage and direct current in the input direction IF the voltage on the capacitive energy storage device is higher than the desired output voltage and the desired outcome is to charge the device,
g) configuring a bidirectional buck/boost converter to stop outputting power if the voltage on the capacitive energy storage device falls below a predetermined level,
h) configuring a bidirectional buck/boost converter to stop inputting power if the voltage on the capacitive energy storage device exceeds a predetermined level,
i) repeating steps (a) through (f) as necessary.

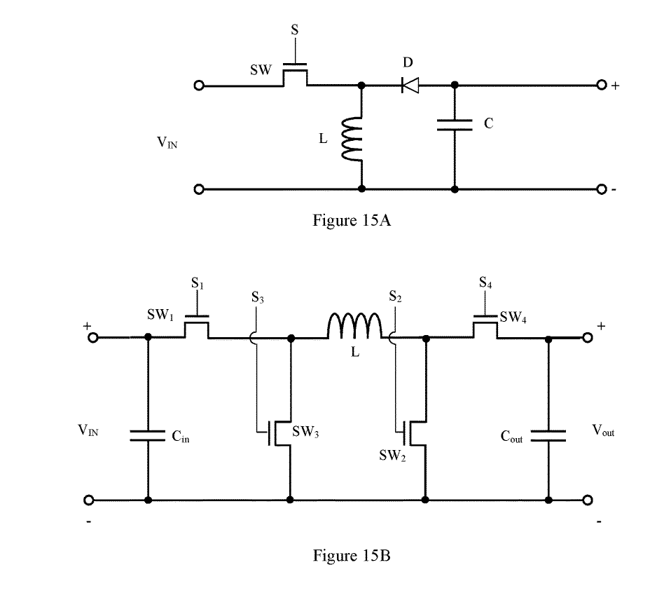

The specifics of operation of the control board 4 are somewhat dependent on the type of buck/boost converter(s) used in the DC-voltage conversion device 3. For example, a buck/boost converter may be a single switch converter of the type shown in FIG. 15A. This type of converter includes a high-side switch SW having an input side coupled to the input voltage Vᵢₙ and an output side coupled to one side of an inductor L, the other side of which is connected to the ground or common voltage (−). A capacitor C is coupled across the output voltage Vₒᵤₜ. A pulsed switching signal S turns the switch on and off. The output voltage depends on the duty cycle of the switching signal S. By way of example, the switches may be implanted as gated switch devices, e.g., MOSFET devices, stacked MOSFET devices, IGCT devices, high drain-source voltage SiC MOSFET devices, and the like depending on the voltage and/or current requirements of the DC-voltage converter for the energy storage cell. In the case of gated switching devices, the control board provides the signals to the gate terminals of the switching devices. The control board 4 can configure this type of buck/boost converter to buck or boost by adjusting the duty cycle of the switching signal S.

FIG. 15B shows an alternative four-switch buck/boost converter. In this type of converter, a first switch SW1 is connected between the high side (+) of the input voltage Vᵢₙ and an input side of the inductor L, a second switch SW2 is connected between an output side of the inductor L and the common voltage (−), a third switch SW3 is connected between the input side of the inductor L and the common voltage, and a fourth switch SW4 is connected between the output side of the inductor and the high side (+) of the output voltage Vₒᵤₜ. An input capacitor C-ᵢₙ may be coupled across the input voltage Vᵢₙ and an output capacitor Cₒᵤₜ may be coupled across the output voltage Vₒᵤₜ.

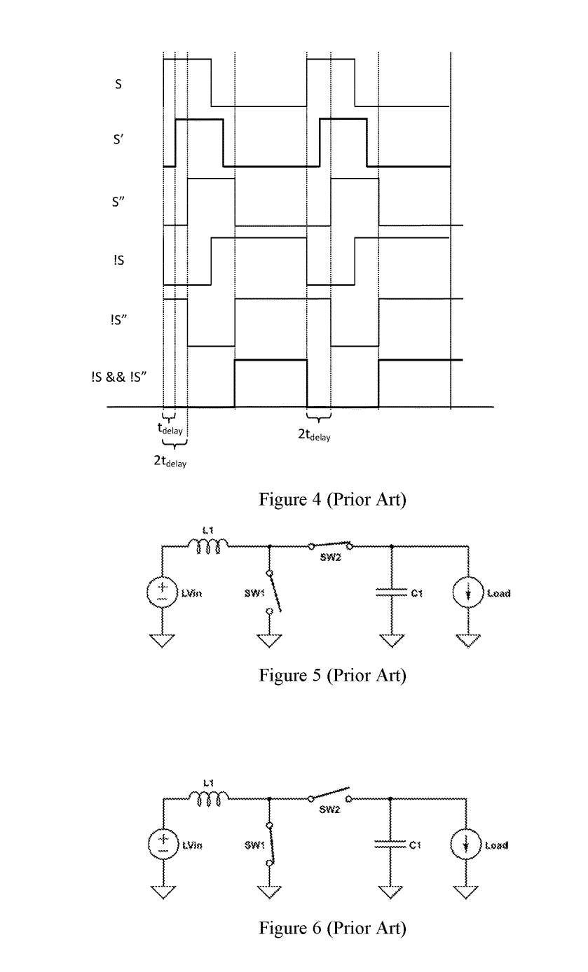

The switches SW1, SW2, SW3, and SW4 change between open (non-conducting) and closed (conducting) states in response to switching signals from the control board 4. To operate in buck mode, the second switch SW2 is open and the fourth switch SW4 closed and pulsed buck mode switching signals are applied to the first switch SW1 and third switch SW3, e.g., as described above with respect to FIG. 3 and FIG. 4. The control board 4 can adjust the output voltage Vₒᵤₜ in buck mode by adjusting the duty cycle signal of the switching signals S1 and S3. To operate in boost mode, the first switch SW1 is open, the third switch SW3 is closed and pulsed boost mode switching signals are applied to the second switch SW2 and fourth switch SW4, e.g., as described above with respect to FIG. 5 and FIG. 6. The control board 4 can adjust the output voltage Vₒᵤₜ in boost mode by adjusting the duty cycle signal of the switching signals S2 and S4.

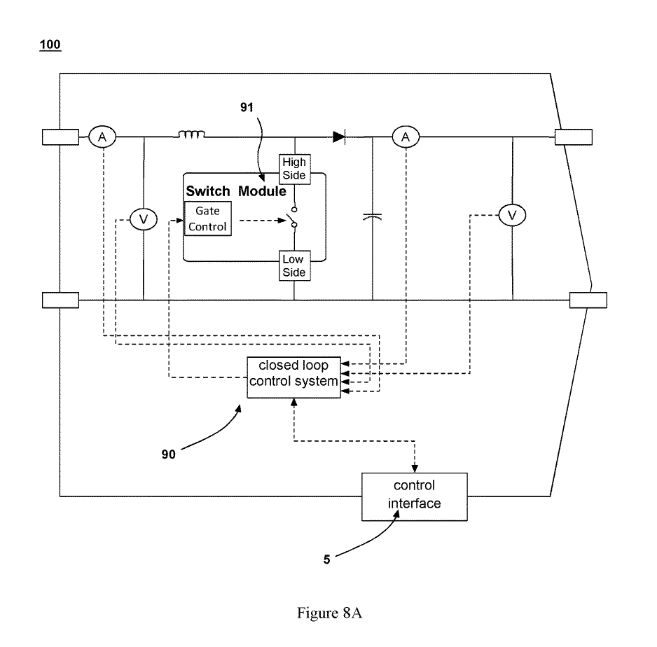

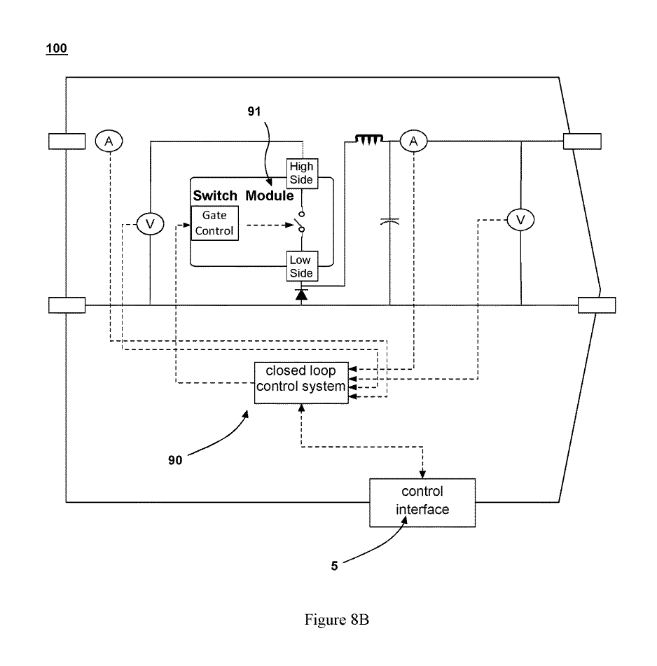

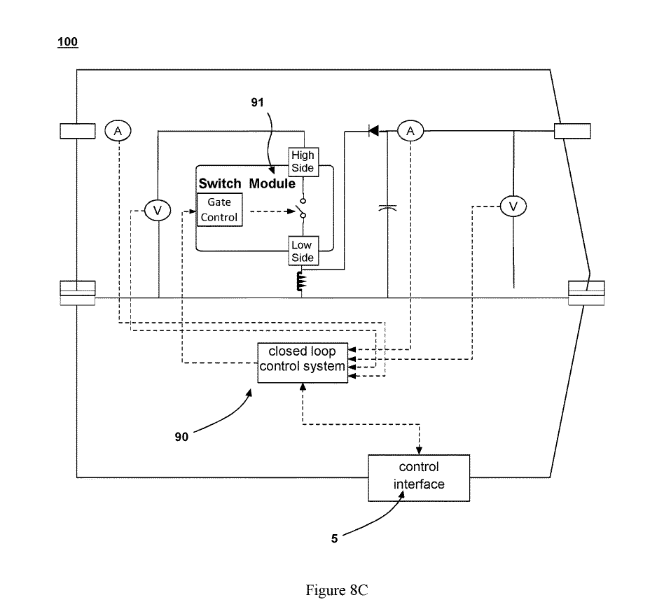

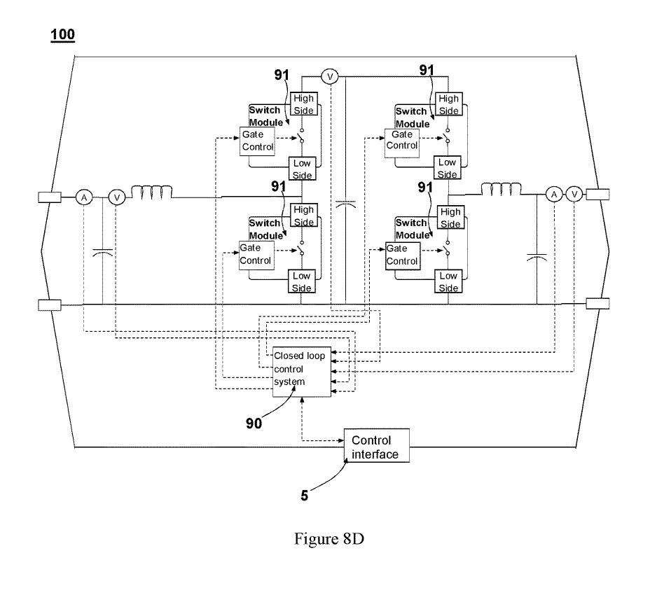

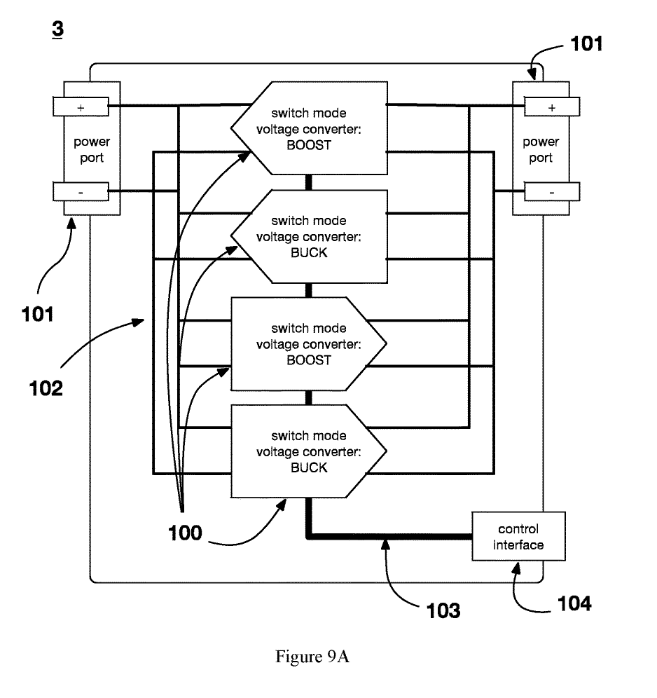

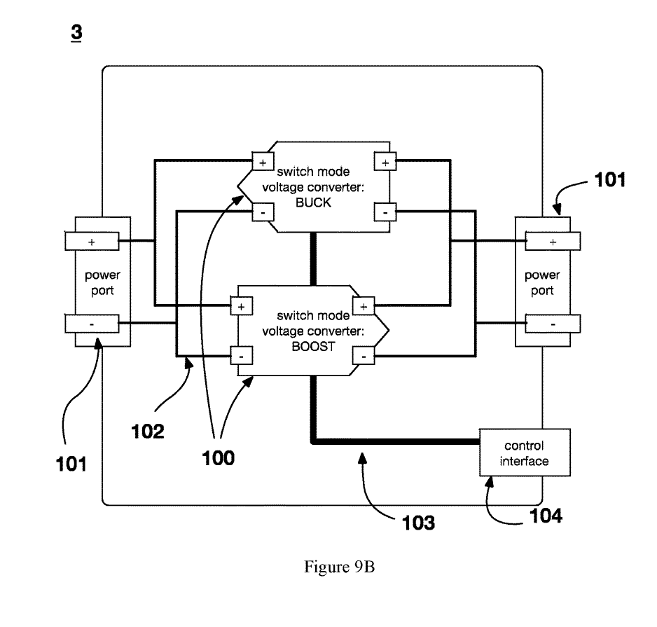

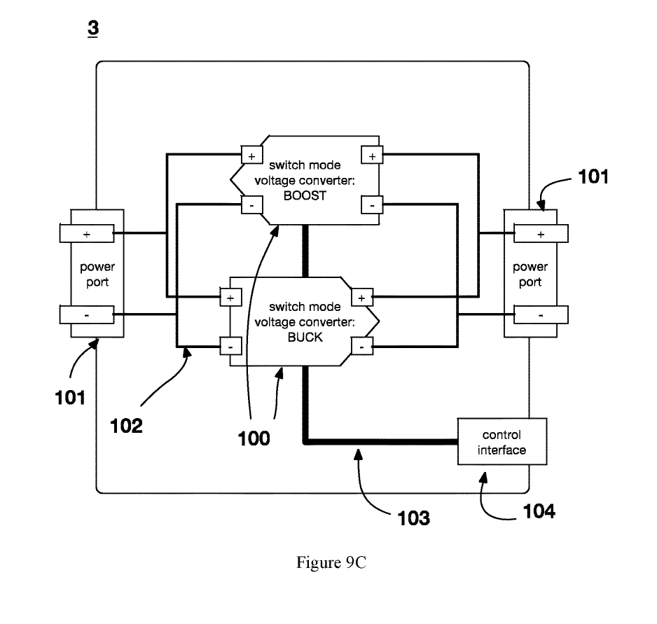

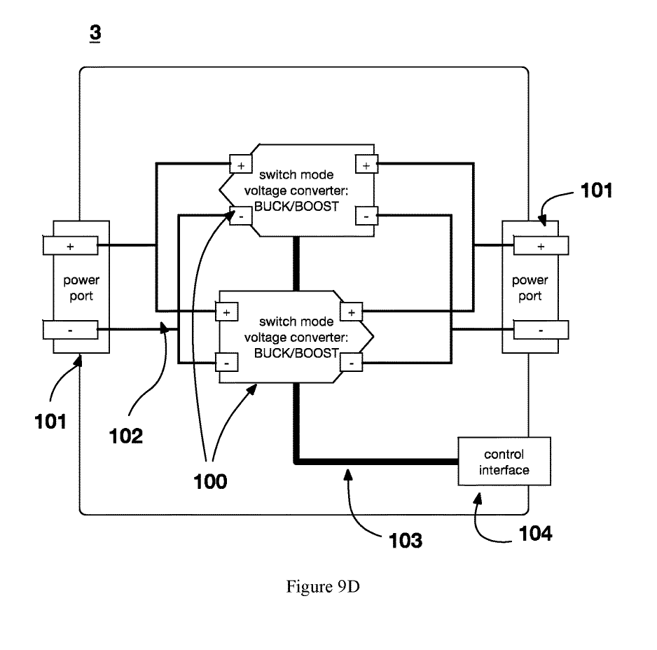

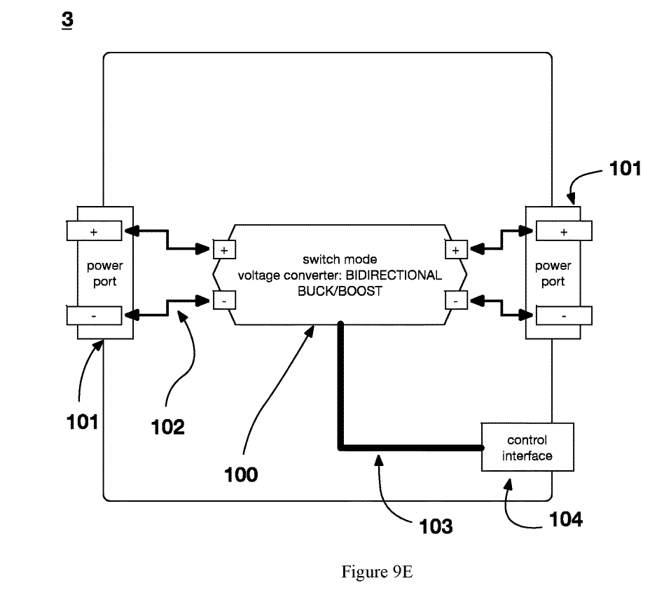

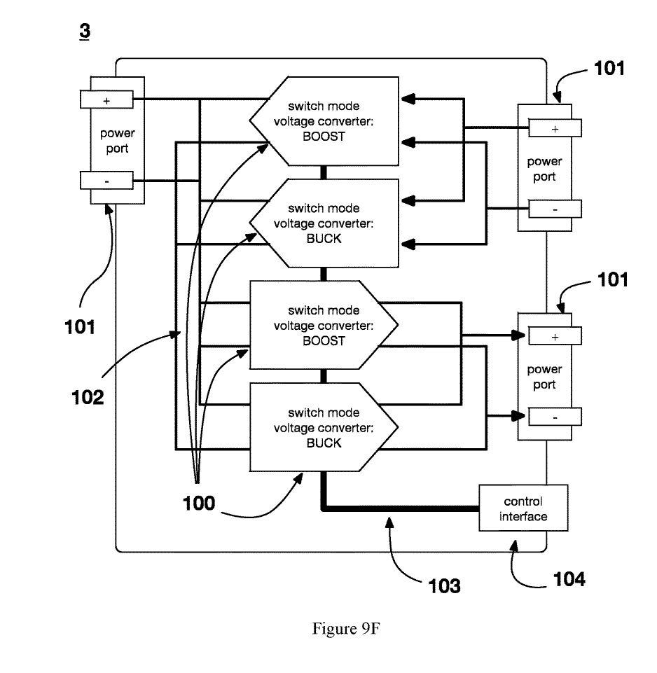

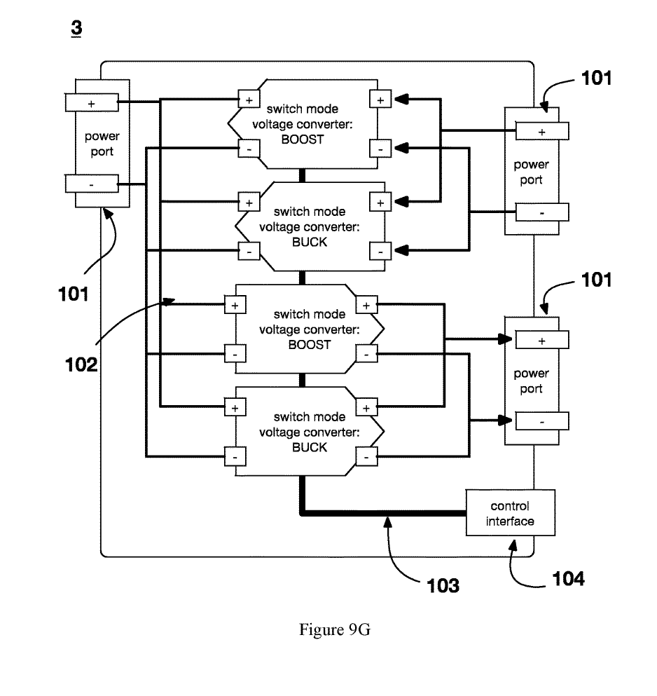

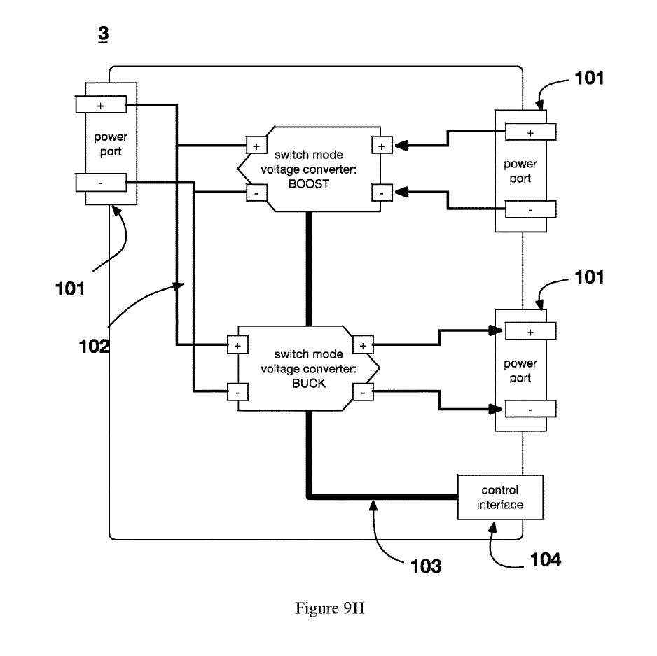

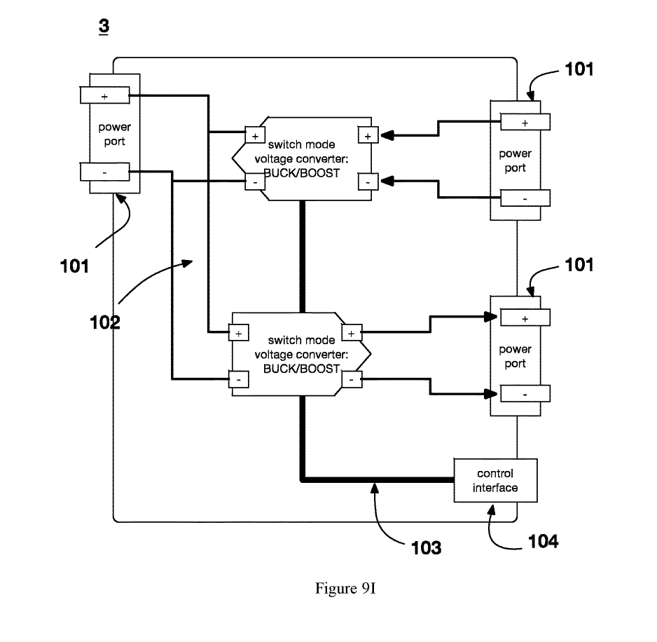

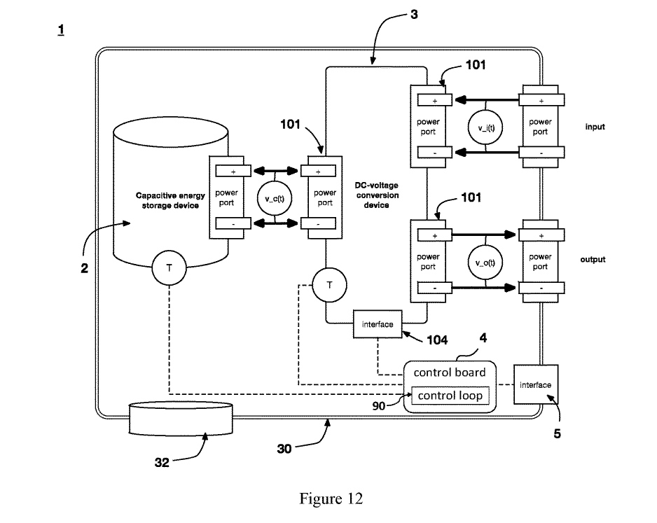

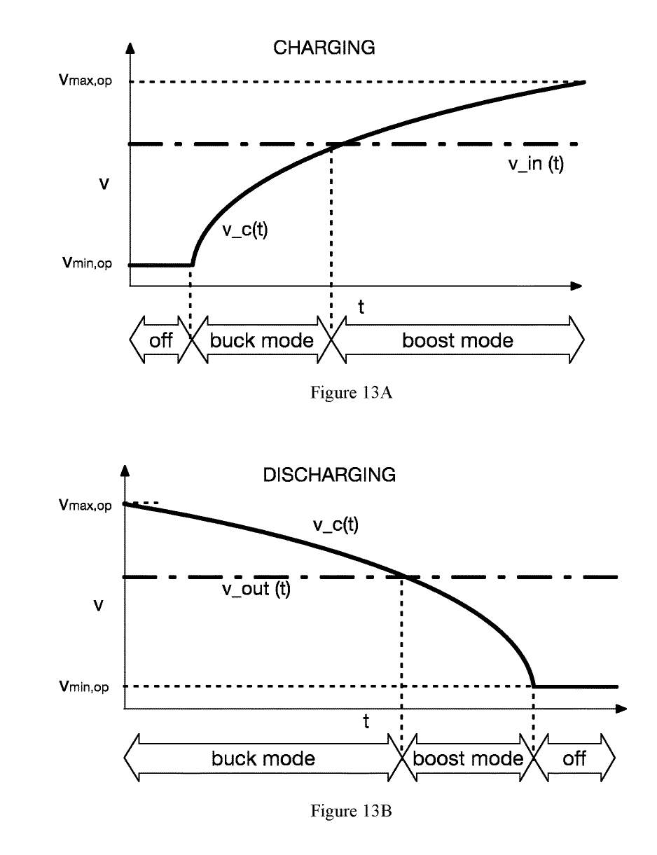

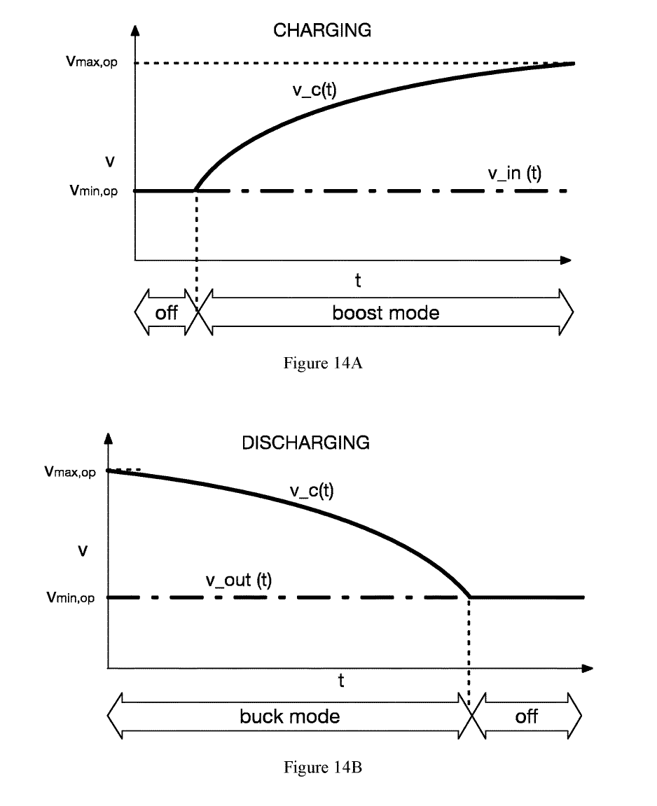

By way of example and not by limitation, the DC-voltage conversion device 3 as depicted in FIGS. 9A, 9B, 9C, 9D, 9E, 9F, 9G, 9H, 9I may include one or more switch-mode voltage converters 100, arranged to boost/or buck the input/output voltages as necessary to achieve the charge and discharge modalities depicted in FIGS. 13A, 13B, 14A and 14B corresponding to the voltage labels v_c(t), v_i(t) and v_o(t) on the capacitive energy storage cell 3 of FIGS. 11 and 12. As shown in FIGS. 9F, 9G, 9H, 9I, the input/output port may be split into a separate input and output. These separate inputs and outputs may have different bus voltages. For example, there may be an input DC bus from a solar inverter which is at a different voltage than an output DC bus meant to transmit power or feed a DC to AC converter. The switch-mode voltage converters 100 may have circuitry selected from the following list: a buck converter (as show in FIG. 8B), boost converter (as show in FIG. 8A), buck/boost converter, bi-directional buck/boost (split-pi) converter (as show in FIG. 8D), Ċuk converter, single-ended primary inductor converter (SEPIC), inverting buck/boost converter (as show in FIG. 8C), or four-switch buck/boost converters.

In FIGS. 9A, 9B, 9C, 9D, 9E, 9F, 9G, 9H, 9I, the switch mode voltage converters 100 are connected to power ports 101, by an interconnect system 102. The power ports 101 include a positive terminal and negative terminal intended to work together to transmit power in either direction. A power port can be an input, output or bidirectional. A control interface 104 is connected to all of the control interfaces on the switch mode voltage converters 100 through a control network 103. The control network may carry target voltages, target currents, observed voltages, observed currents, temperatures and other parameters necessary to control the system. The control network 103, control interfaces 104, control board 4, and control loops 90 may or may not be combined in a single discrete physical package. For example, one implementation may have all aforementioned elements distributed throughout a system and another implementation may contain all elements in a single microprocessor unit.

In one implementation the control board 4 may control the DC-voltage converter 3 in a way that maintains the output voltage of the energy storage cell, e.g., the output voltage of the DC-voltage converter Vₒᵤₜ, at a constant level during a discharge of the meta-capacitor (s) (see, FIGS. 13B and 14B) from an initial charge state ((v_c(t)) to a minimum charge state (v_c(t)=v_{min,op}) wherein the minimum charge state (v_{min,op}), is defined by a voltage on the meta-capacitor (s) which corresponds to the residual energy equal to from 0% to 20% of the initial reserved energy, where the reserved energy of the meta-capacitor (s) can be calculated by E=1/2CV² where E is energy, C is capacitance, and V is voltage. In implementations where the control board 4 is a programmable device, the constant output voltage of the energy storage cell can be a programmable value.

In still another implementation of the energy storage cell, wherein the output voltage is made constant by the DC-voltage conversion device selected from the list comprising a buck regulator, a boost regulator, buck and boost regulators with separate input/outputs, bi-directional boost/buck regulator, split-pi converter.

In some implementations, the cell 1 includes circuitry configured to enable observation of parameters selected from the following list: the voltage on the meta-capacitor, the current going into or out of the meta-capacitor, the current flowing into or out of the DC-voltage conversion device, the output voltage of the DC-voltage conversion device, the temperature at one or more points within the meta-capacitor, the temperature at one or more points within the DC-voltage conversion device. In another implementation, the energy storage cell further comprises an AC-inverter to create AC output voltage, wherein the DC output voltage of the DC-voltage conversion device is the input voltage of the AC-inverter. In yet another implementation, energy storage cell further comprises power electronics switches which are based on Si insulated-gate bipolar transistors (IGBTs), SiC MOSFETs, GaN MOSFETs, graphene or comprising organic molecular switches. In one embodiment of the energy storage cell, the power electronics switches comprise multiple switch elements stacked in series to enable switching of voltages higher than the breakdown voltage of individual switch components.

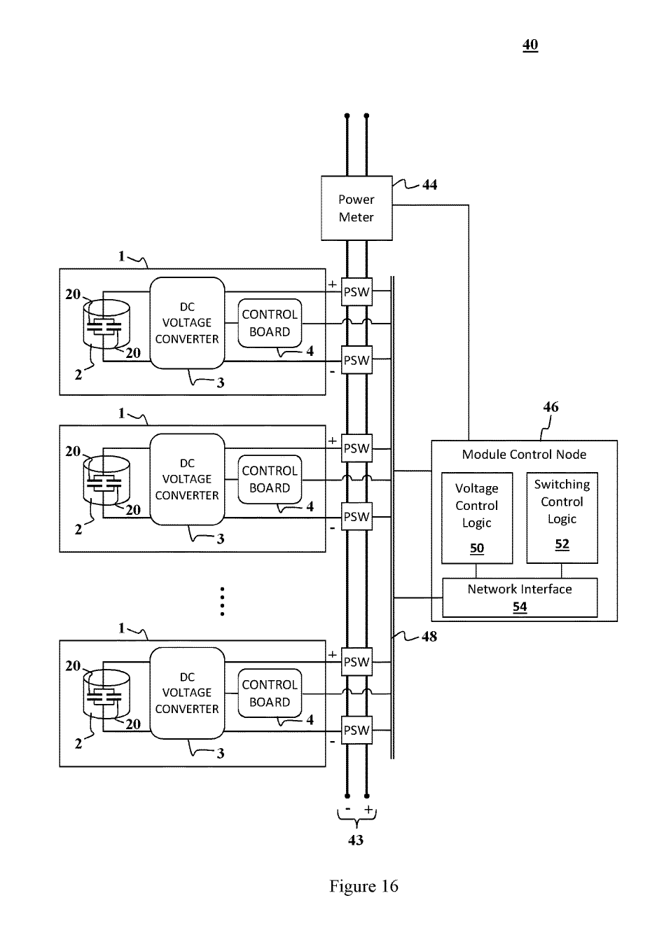

In another aspect of the present disclosure, a capacitive energy storage module 40, e.g., as illustrated in FIG. 16. In the illustrated example, the energy storage module 40 includes two or more energy storage cells 1 of the type described above. Each energy storage cell includes a capacitive energy storage device 2 having one or more meta-capacitors 20 and a DC-voltage converter 3, which may be a buck converter, boost converter, or buck/boost converter. In addition, each module may include a control board 4 of the type described above with respect to FIGS. 10, 11, 12, and an (optional) cooling mechanism (not shown). The module 40 may further include an interconnection system that connects the anodes and cathodes of the individual energy storage cells to create a common anode and common cathode of the capacitive energy storage module.

In yet another aspect, some implementations, the interconnection system includes a parameter bus 43 and power switches PSW. Each energy storage cell 1 in the module 40 may be coupled to the parameter bus 43 via the power switches PSW. These switches allow two or more modules to be selectively coupled in parallel or in series via two or more rails that can serve as the common anode and common cathode. The power switches can also allow one or more energy storage cells to be disconnected from the module, e.g., to allow for redundancy and/or maintenance of cells without interrupting operation of the module. The power switches PSW may be based on solid state power switching technology or may be implemented by electromechanical switches (e.g., relays) or some combination of the two.

In some implementations, the energy storage module further comprises a power meter 44 to monitor power input or output to the module. In some implementations, the energy storage module further comprises a networked control node 46 configured to control power output from and power input to the module. The networked control node 46 allows each module to talk with a system control computer over a high speed network. The networked control node 46 includes voltage control logic circuitry 50 configured to selectively control the operation of each of voltage controller 3 in each of the energy storage cells 2, e.g., via their respective control boards 4. The control node 46 may also include switch control logic circuitry 52 configured to control operation of the power switches PSW. The control boards 4 and power switches PSW may be connected to the control node 46 via a data bus 48. The voltage control and switching logic circuitry in the networked control node 46 may be implemented by one or more microprocessors, microcontrollers, application specific integrated circuits (ASICs), field programmable gate arrays (FPGAs), or complex programmable logic devices (CPLDs). The control node 46 may include a network interface 54 to facilitate transfer of signals between the voltage control logic circuitry 50 and the control boards 4 on the individual energy storage cells 2 and also to transfer signals between the switching logic circuitry 52 and the power switches PSW, e.g., via the data bus 48.

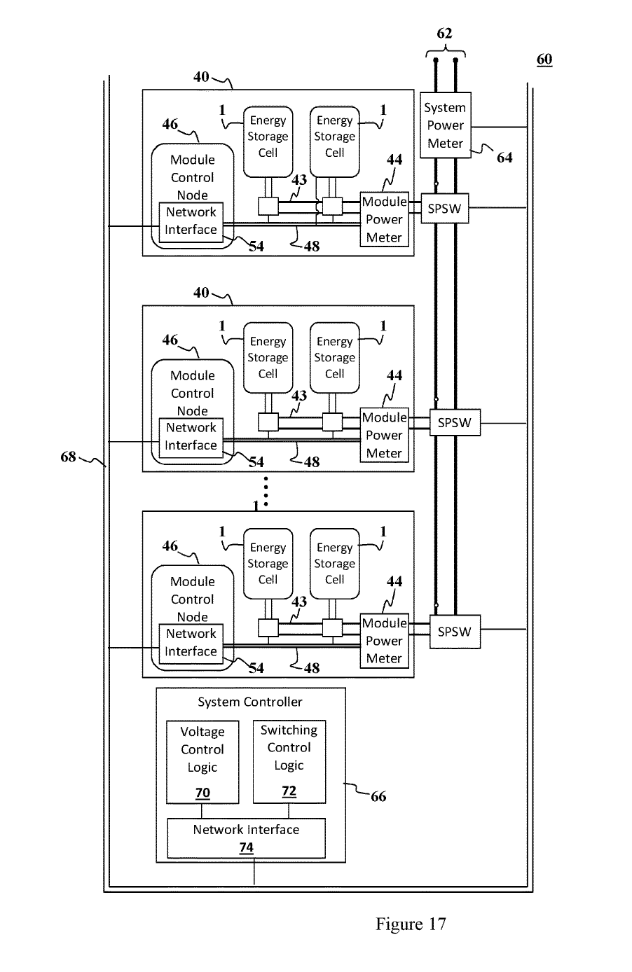

According to yet another aspect of the present disclosure a capacitive energy storage system may include two or more networked capacitive energy storage modules, e.g., of the type shown in FIG. 16. One embodiment of such a capacitive energy storage system 60 is shown in FIG. 17. The system 60 includes two or more energy storage modules 40 of the type shown in FIG. 16. Each capacitive energy storage module 40 includes two or more capacitive energy storage cells 1, e.g., of the type shown in FIGS. 10, 11, 12, connected by an interconnection system 43 and controlled by a control node 46. Each capacitive energy storage module may also include a module power meter 44. Although it is not shown in FIG. 16, each control node 46 may include voltage control logic circuitry 50 to control voltage controllers within the individual capacitive energy storage cells 1 and switching logic circuitry 52 to control internal power switches with the module, as described above. In addition, each control node 46 includes an internal data bus 48 and a network interface 54, which may be connected as described above. Power to and from capacitive energy storage modules 40 is coupled to a system power bus 62 via system power switches SPSW, which may be based on solid state power switching technology or may be implemented by electromechanical switches (e.g., relays) or some combination of the two. In some implementations, there may be an inverter (not shown) coupled between each capacitive energy storage module 40 and the system power bus 62 to convert DC power from the module to AC power or vice versa.

The system 60 includes a system controller 66 connected to a system data bus 68. The system controller may include switching control logic 70, voltage control logic 72, and system network interface 74. The voltage control logic 70 may be configured to control the operation of individual DC-voltage controllers within individual cells 1 of individual modules 40. The switching control logic 72 may be configured to control operation of the system power switches SPSW and also the power switches PSW within individual capacitive energy storage modules 40. Voltage control signals may be sent from the voltage control logic 72 to a specific DC-voltage control device 3 within a specific capacitive energy storage cell 1 of a specific capacitive energy storage module through the network interface 74, the system data bus 68, the module network interface 54 of the control node 46 for the specific module, the module data bus 48, and the control board 4 of the individual cell 1.

By way of example, and not by way of limitation, the system controller 66 may be a deterministic controller, an asynchronous controller, or a controller having distributed clock. In one particular embodiment of the capacitive energy storage system 60, the system controller 66 may include a distributed clock configured to synchronize several independent voltage conversion devices in one or more capacitive energy storage cells of one or more of the capacitive energy storage modules 40.

In all embodiments described, as well as all alterations, substitutions, and derivatives apparent to those skilled in the art, the capacitive energy storage system is configurable to be connected to one or more of a power generation system, a grid, and a load.

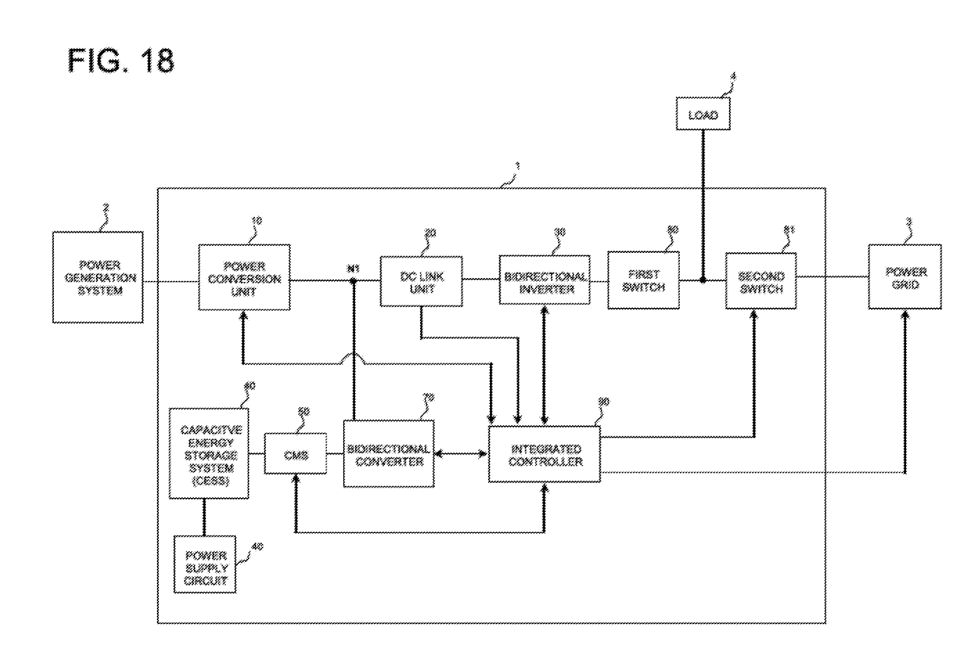

Referring to FIG. 18, as a non-limiting example, the capacitive energy storage system supplies power to a load 104 by being coupled to a power generation system 102 and a grid 103

The power generation system 102 is a system that generates power by using an energy source. The power generation system 102 supplies the generated power to the capacitive energy storage system. The power generation system 102 may be a solar power generation system, a wind power generation system, or a tidal power generation system. However, the present embodiment is not limited thereto, and the power generation system 102 may be any suitable power generation system that may generate power by using renewable energy such as solar heat or geothermal heat, or by using any other suitable energy sources. In one embodiment, solar cells for generating electrical energy by using sunlight may be applied to the capacitive energy storage system, and the solar cells may be distributed at PV farms, houses, and factories because it is easy to install the solar cell thereon. The power generation system 102 may act as a high-capacity energy system by generating power by using a plurality of power generation modules that are arranged in parallel.

The grid 103 includes a power plant, a substation, power lines, and the like. When the grid 103 is in a normal state, the grid 103 supplies power to the capacitive energy storage system and/or the load 104, or receives power supplied from the capacitive energy storage system. This allows for energy arbitrage and peak load demand shaving. When the grid 103 is in an abnormal state, the grid 103 does not supply power to either the capacitive energy storage system or the load 104, and the capacitive energy storage system stops supplying power to the grid 103.

The load 104 consumes power generated by the power generation system 102, power stored in a capacitive energy storage system, or power supplied from the grid 103. A factory or housing complex may be an examples of the load 104.

The capacitive energy storage system may store energy generated by the power generation system 102 and send the generated power to the grid 103. The capacitive energy storage system may deliver stored energy to the grid 103 or store energy supplied from the grid 103. In an abnormal situation, for example, when there is a power failure in the grid 103, the capacitive energy storage system may supply energy to the load 104 by performing as an uninterruptible power supply (UPS). Even when the grid 103 is in a normal state, the capacitive energy storage system may supply power generated by the power generation system 102 or supplied from the grid 103 to the load 104 or the grid.

The capacitive energy storage system can be connected to a direct current (DC) link unit 120 and a bidirectional inverter 130. A power converting unit 110 can be coupled between the power generation system 102 and a first node N1, and delivers power generated by the power generation system 102 to the first node N1. Here, an output voltage of power output from the power converting unit 110 can be converted into a DC link voltage and supplied to a load or a grid via the DC link unit 120. That is, the power generated by the power generation system 102 may be supplied to the capacitive energy storage system, a grid, or a load by operating the power converting unit 110.

The power converting unit 110 may include a converter or a rectifier circuit according to the type of the power generation system 102. More specifically, if the power generation system 102 generates DC power, the power converting unit 110 may include a voltage converter for converting the DC voltage to a different DC voltage. On the contrary, if the power generation system 102 generates alternating current (AC) power, the power converting unit 110 may include an AC to DC converter. In particular, if the power generation system 102 is a solar power generation system, the power converting unit 110 may include a maximum power point tracking (MPPT) converter so as to obtain maximum power output from the power generation system 102 according to a change in solar radiation, temperature, or the like.

When the power generation system 102 generates no power, the power converting unit 110 may stop operating and reduce or minimize power consumption of a converter included in the power converting unit 110 or the like.

The DC link unit 120 is coupled between the first node N1 and the bidirectional inverter 130 and maintains the DC link voltage of the first node N1. A level of a voltage at the first node N1 may become unstable due to an instantaneous voltage drop of the power generation system 102 or the grid 103 or a peak load occurrence in the load 104. However, the voltage at the first node N1 needs to be stabilized to normally operate the bidirectional inverter 130 and the bidirectional converter 170. The DC link unit 120 may be included to stabilize a level of the DC link voltage of the first node N1, and may be realized by, for example, a suitably large capacitor (e.g., a mass storage capacitor), etc. Although the DC link unit 120 is connected to the capacitive energy storage system separate from other parts in the embodiment shown in FIG. 18, the DC link unit 120 may be included in the power converting unit 10, the bidirectional inverter 130, or the bidirectional converter 170.

The bidirectional inverter 130 is a power converter coupled between the DC link unit 120 and the second switch 180. The bidirectional inverter 130 converts the DC link voltage Vₗᵢₙₖ output from the power generation system 102 or into an alternating current (AC) voltage of the grid 103 and outputs the AC voltage in a discharging mode. The bidirectional inverter 130 rectifies an AC voltage output from the grid 103 into the DC link voltage to be stored in a charging mode. The bidirectional inverter 130 may include a filter for removing harmonics from the AC voltage output to the grid 103, and a phase-locked loop (PLL) circuit for matching a phase of the AC voltage output from the bidirectional inverter 130 to a phase of the AC voltage of the grid 103 in order to prevent generation of reactive power. Also, the bidirectional inverter 130 may perform other functions such as restriction of voltage variation range, power factor correction, removal of DC components, and protection from transient phenomenon. When it is unnecessary for supplying the power generated by the power generation system 102 or the power stored to the grid 103 or the load 104, or when power from the grid 103 is unnecessary for charging, the operation of the bidirectional inverter 130 may be stopped so as to minimize or reduce power consumption.

The capacitive energy storage system receives and stores power generated by the power generation system 102 or power output from the grid 103, and supplies power stored to the load 104 or the grid 103.

Referring to FIG. 18, where the capacitive energy storage system is connected to a power generation system, a grid, and a load, the second switch 180 and the first switch 181 are coupled in series, and the second switch 180 is between the bidirectional inverter 130 and a second node N2. The second switch 180 and the first switch 181 control the flow of current between the power generation system 102 and the grid 103 by being turned on or off under the control of the integrated controller 190. Likewise, second switch 180 and third switch 182 are coupled in series, and the second switch 180 is between the bidirectional inverter 130 and the second node N2. The second switch 180 and the third switch 182 control the flow of current between the power generation system 102 and the load 104 by being turned on or off under the control of the integrated controller 190. The second switch 180 and the first switch 181 may be turned on or off according to various states of the power generation system 102, the grid 103, and the capacitive energy storage system. For example, when power required by the load 104 is high, both the second switch 180 and the first switch 181 may be turned on to use power from the power generation system 102 and the grid 103. If power required by the load 104 is greater than available power supplied from the power generation system 102 and the grid 103, power stored in the capacitive energy storage system may also be supplied to the load 104.

Switch 180 and 181 enable power from a power generation unit (PGU) and capacitive energy storage system to flow to the grid and from grid to CESS. Switch 182 stops from going to load. Switch 180 stops from PGU and allows grid to power load. For example, if there is a power failure in the grid 103, the first switch 181 is turned off and the second switch 180 is turned on. Accordingly, power from the power generation system 102 or the capacitive energy storage system may be supplied to the load 104, but does not flow into the grid 103, thereby preventing a worker who works at a power distribution line of the grid 103 or the like from getting an electric shock. If the power needed by the grid and the load exceeds the power generation system's ability to supply either the second switch 180 or the third switch 182 may cut power delivered to the gird or load, respectively.

It should be noted that this is but one possible embodiment and the scope of the invention is intended to include all cases where the system is connected to all possible combinations of multiple loads, grids, and power generation systems, each of which could have their own independent corresponding switches that functioned independently of each other.

The integrated controller 190 monitors the states of the power generation system 102, the grid 103, the capacitive energy storage system, and the load 104, and controls the power converting unit 110, the bidirectional inverter 130, the second switch 180, and the first switch 181 according to results of the monitoring. The integrated controller 190 monitors whether the grid 103 is coupled to the load 104, whether the power generation system 102 generates power, and the like. Furthermore, the integrated controller 190 may monitor an amount of power generated by the power generation system 102, a charge state of the capacitive energy storage system, an amount of power consumed by the load 104, time, and the like. In some implementations, the integrated controller 190 may be in communication with an external central controller to manage charging and discharging of the capacitive energy storage system (CESS). Alternatively, the integrated controller may operate the CESS charging and discharging using an algorithm to sell energy to the grid utility operators and buy energy to charge the CESS from the grid utility operators.

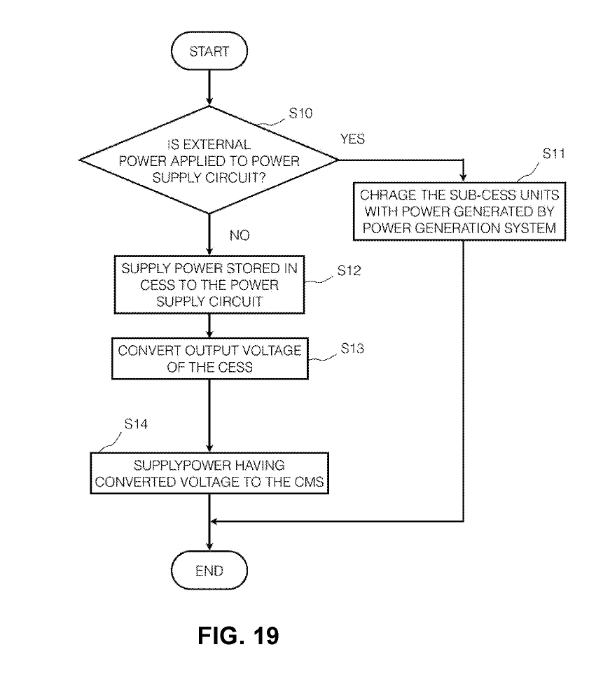

FIG. 19 is a flowchart illustrating a method of controlling the system power meter 62, according to an embodiment of the present invention.

Referring to FIG. 19 the system controller 66 determines whether the external power is applied to the system power meter 62 (operation S10). If the system controller 66 determines that the external power is applied to the system power meter 62, since it is a normal state, the system controller 66 applies the external power to the system controller (operation S11).

If the system controller determines that the external power is not applied to the system power meter 62, the power switching unit 61 supplies power stored in the capacitive energy storage system to the integrated controller and the bidirectional inverter via the DC link unit according to the control of the system controller (operation S12). The bidirectional inverter converts the voltage of the power output from the capacitive energy storage system into a voltage with a previously set value (operation S13). The previously set value may be a voltage value of the external power or a voltage value for operating the parts included in the system controller.

The power having the converted voltage is supplied to the system controller (operation S14), thus stably supplying an operating power to the system controller even in an abnormal state when the external power is not supplied.

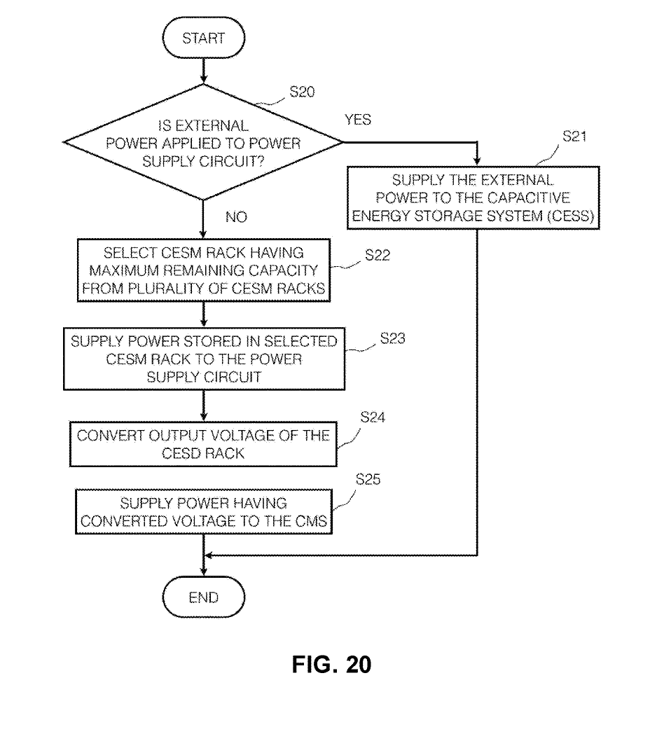

FIG. 20 is a flowchart illustrating a method of controlling the system power meter 62, according to another embodiment of the present invention.

Referring to FIG. 20, the system controller determines whether the external power is applied to the system power meter 62 (operation S20). If the system controller determines that the external power is applied to the system power meter 62, since it is a normal state, the system controller applies the external power to the system controller (operation S21).

If the system controller determines that the external power is not applied to the system power meter 62, one of the capacitive system modules having the maximum remaining capacity is selected (operation S22). One of the capacitive system modules having the maximum remaining capacity is selected automatically, or the system controller selects a specific module among the modules. The power switching unit 61 supplies power stored in the selected module (operation S23). The module converts a voltage of the power output from the capacitive energy storage system into a voltage with a previously set value (operation S24). The previously set value may be a voltage value of the external power or a voltage value for operating the parts included in the system controller.

The power having the converted voltage is supplied to the system controller (operation S25), thus stably supplying an operating power to the system controller even in an abnormal state when the external power is not supplied.

A program for executing the methods according to the embodiments of the present invention in the capacitive energy storage systems according to the embodiments of the present invention may be stored in a recording medium. The recording medium is a medium that may be read by a processor or a computing device. The recording medium may be a semiconductor recording medium (e.g., a flash memory, a static random access memory (SRAM), or the like). For example, the recording medium may be embedded in the system controller or the integrated controller 190, and the program may be executed by a processor, for example, the integrated controller 190.

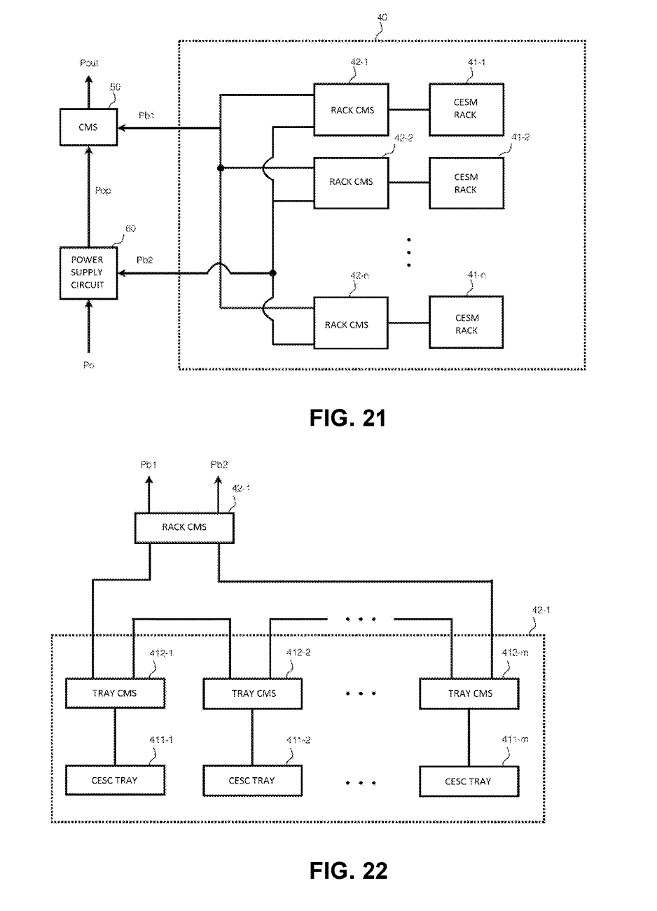

An example of the construction of an alternative capacitive energy storage system (CESS) 40 is described in more detail with reference to FIG. 21 and FIG. 22.

FIG. 21 is a block diagram of the capacitive energy storage system (CESS) 40, the capacitive energy storage module (CESM) management system (CMS) 50, and the power supply circuit 60 that are coupled to each other, according to an embodiment of the present invention. FIG. 3 is a block diagram of one or more CESM racks 41-1 . . . 41-n according to an embodiment of the present invention.

Referring to FIGS. 21 and 22, the CESS 40 may include the one or more CESM racks 41-1 . . . 41-n and one or more rack CMSs 42-1 . . . 42-n that respectively control the CESM racks 41-1 . . . 41-n. The CESM racks 41-1 . . . 41-n may include a plurality of capacitive energy storage cell (CESC) trays 411-1 . . . 411-m and a plurality of tray CESC management systems (CMSs) 412-1 . . . 412-m that respectively control the CESC trays 411-1 . . . 411-m.

Each of the CESC trays 411-1 . . . 411-m may include one or more CESC. The CESC may include one or more meta-capacitors, e.g., as described in U.S. patent application Ser. No. 15/043,315 to Ian Kelly Morgan et al, filed Feb. 12, 2016, which may include a meta-dielectric material between two electrodes, e.g., as described in U.S. patent application Ser. No. 15/043,246 to Barry K. Sharp et al., filed Feb. 12, 2016, U.S. patent applications Ser. Nos. 15/043,186 and 15/043,209 to Paul Furuta et al, filed Feb. 12, 2016, all of which applications are incorporated herein by reference, any other type of capacitive energy storage device described elsewhere, or the like. The CESC included in the CESC trays 411-1 . . . 411-m may be coupled to each other in series, in parallel, or in combination thereof. Furthermore, the one or more CESC trays 411-1 . . . 411-m may be coupled to each other in series. However, the present embodiment is not limited thereto, and the one or more CESC trays 411-1 . . . 411-m may be coupled to each other in parallel or in combination of parallel and series.

Some examples illustrating possible the construction and the operation of the power supply circuit 60 will be described in more detail below.

### First Implementation

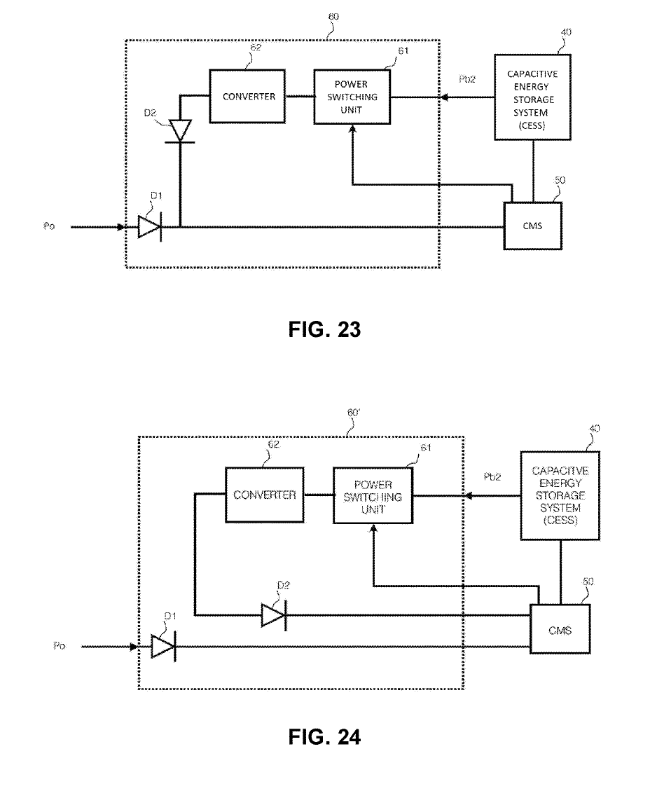

FIG. 23 is a circuit diagram illustrating the power supply circuit 60 according to an aspect of the present disclosure.

Referring to FIG. 23, the power supply circuit 60 may include a first diode D1, a second diode D2, a power switching unit 61, and a converter 62.

The power supply circuit 60 receives the external power Po as an operating power of the CMS 50 and supplies the external power Po to the CMS 50. The power supply circuit 60 includes a path for supplying the external power Po to the CMS 50, and includes the first diode D1 coupled between an input terminal to which the external power Po is applied and the CMS 50 on the path for supplying the external power Po so as to prevent a back-flow of current.

Also, the power supply circuit 60 receives the power Pb2 output from the CESS 40 as the operating power of the CMS 50 and supplies the power Pb2 to the CMS 50. The power supply circuit 60 includes a path for supplying the power Pb2 to the CMS 50, and includes the second diode D2, the power switching unit 61, and the converter 62 on the path for supplying the power Pb2.

The power switching unit 61 controls a supply of the power of the CESS 40 to the converter 62. If an external power is not applied to the power supply circuit 60, the power switching unit 61 is turned on and supplies the power of the CESS 40 to the converter 62. An operation of turning on/off the power switching unit 61 may be controlled by a control signal of the CMS 50. For example, if a P-channel field effect transistor (FET) is used as the power switching unit 61, in a normal state when the external power is supplied to the CMS 50, the CMS 50 applies a high level control signal to a gate electrode of the P-channel FET to prevent the power of the CESS 40 from being transmitted to the converter 62. However, in an abnormal state when the external power is not supplied to the CMS 50, the CMS 50 applies a low level control signal to the gate electrode of the P channel FET to supply the power of the CESS 40 to the converter 62.

The converter 62 converts a voltage of the power of the CESS 40 supplied through the power switching unit 61 into a previously set voltage. The previously set voltage may have the same magnitude as a voltage of the external power Po. For example, if the voltage of the external power Po supplied from the outside is 24V, and an output voltage of the CESS 40 is 50V, a voltage drop type DC-DC converter may be used as the converter 62 to convert 50V into 24V that is supplied to the CMS 50. However, the present embodiment is not limited thereto, and the converter 62 may operate as a drop/buck (or step down) voltage converter or a boost (step up) voltage converter according to the voltage of the external power Po and the output voltage of the CESS 40.

The second diode D2 is coupled between the converter 62 and the CMS 50 and prevents a back flow of current on the path for supplying the power Pb2 output from the CESS 40. The second diode D2 may supply power to the CMS 50 through the same terminal as a terminal for supplying the external power Po to the CMS 50. That is, cathode electrodes of the first diode D1 and the second diode D2 may be coupled to each other.

In FIG. 23, when the external power Po is not supplied, the power supply circuit 60 supplies the power Pb2 output from the CESS 40 to the CMS 50, thereby stably operating the CMS 50.

### Second Implementation

FIG. 24 is a circuit diagram illustrating a power supply circuit 60′ according to another aspect of the present disclosure. For example, the power supply circuit 60′ may be used to replace the power supply circuit 60 of FIG. 18.

Referring to FIG. 23, the power supply circuit 60′ may include the first diode D1, the second diode D2, the power switching unit 61, and the converter 62. The operations of the elements of the power supply circuit 60′ are substantially the same as corresponding components described with reference to FIG. 4, and thus differences there between will now be described.

The converter 62 of the present embodiment converts a voltage of the power of the CESS 40 supplied through the power switching unit 61 into a voltage for operating the parts included in the CMS 50. The CMS 50 may include a regulator to convert a voltage applied from the outside into the voltage for operating the parts included therein. For example, the CMS 50 receives external power having a voltage of 24V and converts the voltage into 5V by using the regulator. Thus, in the present embodiment, if an output voltage of the CESS 40 is 50V, and the parts included in the CMS 50 operate at 5V, a voltage drop type DC-DC converter may be used as the converter 62 to convert 50V into 5V and supply the power to the CMS 50. However, the present embodiment is not limited thereto, and the converter 62 may convert the voltage of the power according to types of the parts included in the CMS 50.

In the present embodiment, the voltages of the external power Po and the power Pb2 of the CESS 40 are different from each other, and thus each of the external power Po and the power Pb2 may be applied to different terminals of the CMS 50. Therefore, the second diode D2 may be coupled to a terminal that is different from a terminal of the CMS 50 to which the external power Po is applied and supplies the power Pb2 of the CESS 40 to the CMS 50.

In FIG. 24, when the external power Po is not supplied, the power supply circuit 60 supplies the power Pb2 output from the CESS 40 to the CMS 50, thereby stably operating the CMS 50. The power supply circuit 60 converts the output voltage of the CESS 40 into the voltage for operating the parts included in the CMS 50, thereby reducing the number of voltage conversions.

### Third Implementation

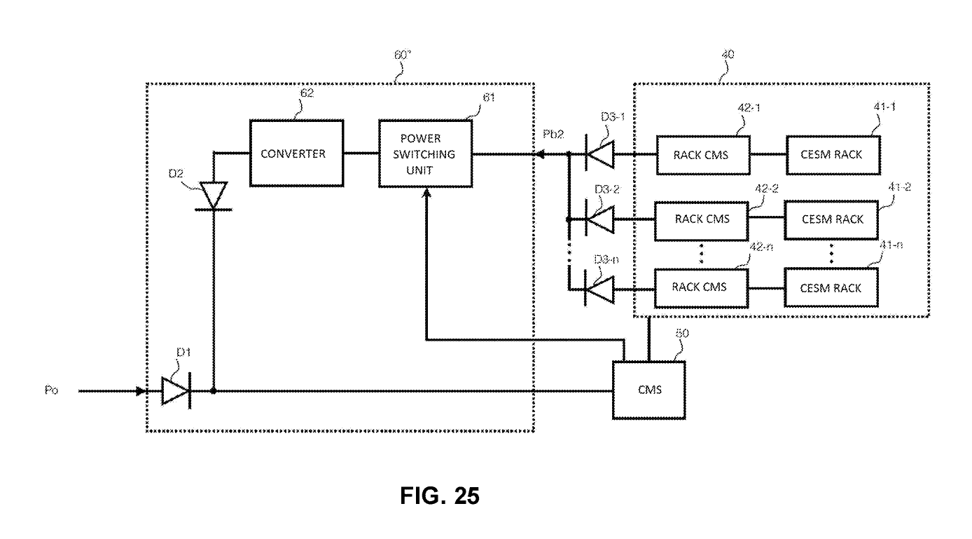

FIG. 25 is a circuit diagram illustrating the power supply circuit 60″ according to another aspect of the present disclosure. For example, the power supply circuit 60″ may be used to replace the power supply circuit 60 of FIG. 1.

Referring to FIG. 25, the power supply circuit 60″ may include the first diode D1, the second diode D2, the power switching unit 61, and the converter 62. The power supply circuit 60 may further include a plurality of diodes D3-1 . . . D3-n for receiving power from one or more power outputs from the CESS 40. The operations of the elements of the power supply circuit 60″ are substantially the same as corresponding components described with reference to FIG. 4, and thus differences there between will now be described.

In the present embodiment, the CESS 40 includes the plurality of CESM racks 41-1 . . . 41-n and the plurality of rack CMSs 42-1 . . . 42-n. The power supply circuit 60 receives power from one or more of the CESM racks 41-1 . . . 41-n having the maximum remaining capacity.

To this end, the CESM racks 41-1 . . . 41-n may include the diodes D3-1 . . . D3-n between output terminals and the power switching unit 61. The greater the remaining capacity of the CESM racks 41-1 . . . 41-n, the higher output voltages are produced. Thus, when the power switching unit 61 is turned on according to the control of the CMS 50, the power is output from one or more of the CESM racks 41-1 . . . 41-n having the maximum remaining capacity and is applied to the power switching unit 61. However, the present invention is not limited thereto. For example, the CMS 50 can communicate various types of data with the rack CMSs 42-1 . . . 42-n, and thus the CMS 50 determines the remaining capacity of the CESM racks 41-1 . . . 41-n in real time, and selects one of the CESM racks 41-1 . . . 41-n from which an operating power of the CMS 50 is received in real time. And, in an abnormal state when the external power is not supplied, the CMS 50 controls the rack CMS of the selected CESM rack to supply the power to the CMS 50.

Alternatively, the power supply circuit 60″ may be previously set to receive the power from a specific CESM rack from among the CESM racks 41-1 . . . 41-n. In this case, the power supply circuit 60″ is coupled to the previously set CESD rack and may receive the power therefrom in an abnormal state.

When the external power Po is not supplied, the power supply circuit 60″ of the present embodiment supplies the power Pb2 output from the CESM 40 to the CMS 50, thereby stably operating the CMS 50. The power supply circuit 60″ receives power from one of the CESM racks 41-1 . . . 41-n having the maximum remaining capacity, thereby performing a cell balancing function, which increases the lifespan of the CESM racks 41-1 . . . 41-n. Alternatively, the power supply circuit 60″ receives the power from a previously set specific CESM rack from among the CESM racks 41-1 . . . 41-n, thereby realizing a relatively simple construction of the power supply circuit 60″.

### Fourth Implementation

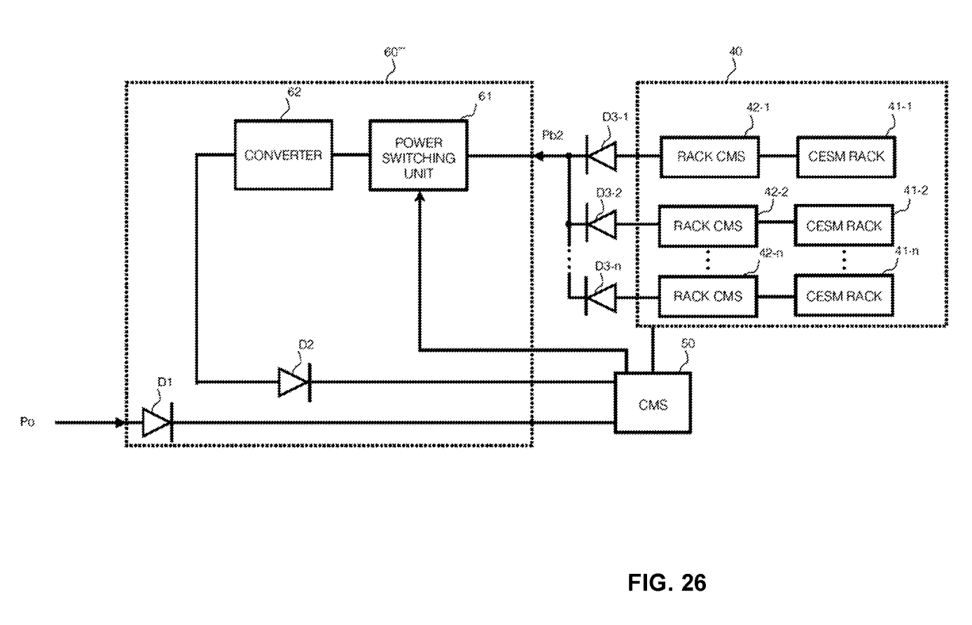

FIG. 26 is a circuit diagram illustrating a power supply circuit 60′ according to another aspect of the present disclosure. For example, the power supply circuit 60′ may be used to replace the power supply circuit 60 of FIG. 1.

Referring to FIG. 26, the power supply circuit 60′ may include the first diode D1, the second diode D2, the power switching unit 61, and the converter 62. The power supply circuit 60′ may further include the diodes D3-1 . . . D3-n for receiving power from one or more power outputs from the CESS 40. The operations of the elements of the power supply circuit 60′ are substantially the same as corresponding components described with reference to FIGS. 5 and 7, and thus the detailed descriptions thereof will not be repeated here.

Aspects of the present disclosure allow for electrical energy storage on a much larger scale than possible with conventional electrical energy storage systems. A wide range of energy storage needs can be met by selectively combining one or more meta-capacitors with a DC-voltage conversion devices into a cell, combining two or more cells into a module, or combining two or more modules into systems.

While the above is a complete description of the preferred embodiment of the present invention, it is possible to use various alternatives, modifications and equivalents. Therefore, the scope of the present invention should be determined not with reference to the above description but should, instead, be determined with reference to the appended claims, along with their full scope of equivalents. Any feature described herein, whether preferred or not, may be combined with any other feature described herein, whether preferred or not. In the claims that follow, the indefinite article “A”, or “An” refers to a quantity of one or more of the item following the article, except where expressly stated otherwise. As used herein, in a listing of elements in the alternative, the word “or” is used in the logical inclusive sense, e.g., “X or Y” covers X alone, Y alone, or both X and Y together, except where expressly stated otherwise. Two or more elements listed as alternatives may be combined together. The appended claims are not to be interpreted as including means-plus-function limitations, unless such a limitation is explicitly recited in a given claim using the phrase “means for.”

What is claimed is:

1. A capacitive energy storage system comprising:
a system power meter;
a system controller; and
at least one energy storage module
wherein said module comprises
at least one energy storage cell
wherein said energy storage cell comprises
a capacitive energy storage device; and
a DC-voltage conversion device;
wherein the capacitive energy storage device comprises one or more metacapacitors,
wherein the output voltage of the capacitive energy storage device is an input voltage of the DC-voltage conversion device during discharging the capacitive energy storage device,
wherein the input voltage of the capacitive energy storage device is an output voltage of the DC-voltage conversion device while charging the capacitive energy storage device,
wherein the capacitive energy storage system is configurable to connect to at least one of the list consisting of a power generation system, a grid, and a load.

2. A capacitive energy storage system comprising:
a system power meter;
a system controller;
at least one or more energy storage modules, wherein each of said one or more energy storage modules includes
at least one energy storage cell, wherein said energy storage cell comprises
a capacitive energy storage device; and
a DC-voltage conversion device;
wherein the capacitive energy storage device comprises one or more metacapacitors,
wherein the output voltage of the capacitive energy storage device is an input voltage of the DC-voltage conversion device during discharging the capacitive energy storage device,
wherein the input voltage of the capacitive energy storage device is an output voltage of the DC-voltage conversion device while charging the capacitive energy storage device, a DC link unit;
a bidirectional inverter; and at least one switch,
wherein the system controller is configurable to control at least one connection to and communication with at least one of the list consisting of a power generation system, a grid, and a load.

3. A capacitive energy storage system as in claim 2 further comprising:
a first switch wherein said first switch is electrically connectable to a grid, and a second switch.

4. A capacitive energy storage system as in claim 2 further comprising:
a power conversion unit,
wherein said power conversion unit is a solar inverter, a maximum power point tracking (MPPT) converter, a DC/DC converter, or an AC/DC converter and can be connected to a power generation system.

5. A capacitive energy storage system as in claim 2 further comprising:
a first switch, and
a second switch wherein said second switch is electrically connectable to a load,
wherein the system power meter is configured to supply external power to the system controller as an operating power of the system controller in a first state in which the external power is applied, and where the system controller is configured to manage power input and power output of the at least one capacitive energy storage modules through the system power meter to a load as the at least partial power demand of the load and system controller in a second state in which the external power is not applied.

6. A capacitive energy storage system as in claim 2 further comprising:
a third switch,
wherein said third switch is between the first switch and the bidirectional inverter
wherein the third switch can electrically isolate the load and grid from the at least one module, power generation unit, and bidirectional inverter.

7. The capacitive energy storage system of claim 6, wherein the load receives power from the grid.

8. A capacitive energy storage system as in claim 2, wherein the system controller is configured to manage charging, discharging, and zero current flow to and from the energy storage media.

9. A capacitive energy storage system as in claims 2 wherein the system power meter is optionally configured to supply power of the capacitive energy storage system to the load and a grid as the operating power of the load and a power source for the grid during a normal state of the grid.

10. A capacitive energy storage system as in claim 2, wherein the metacapacitor is a capacitor comprising a first electrode, a second electrode, and a metadielectric disposed between the first electrode and the second electrode.

11. A capacitive energy storage system as in claim 2, wherein the first electrode and the second electrode are flat and planar and positioned parallel to each other.

12. A capacitive energy storage system as in claim 2, wherein the first electrode and the second electrode are rolled and planar and positioned parallel to each other.

13. A capacitive energy storage system as in claim 2, wherein said metacapacitors are comprised of at least one type of metadielectric materials having a relative permittivity of at least 1000 and resistivity of at least 10¹⁶ ohm cm.

14. A capacitive energy storage system as in claim 13, wherein said metacapacitors are comprised of crystalline metadielectric material comprising at least one type of organic composite compounds, wherein said organic composite compounds have at least one type of enhanced polarizable unit attached to electrically resistive substituents.

15. The composite organic compound of claim 13, wherein the enhanced polarizable unit may consist of ionic polarizable fragments, non-linear electrostatic fragments, and hyperelectronic fragments.

16. The composite organic compound of claim 13, wherein the electrically resistive substituents may consist of structured polycyclic organic fragments, alkyl chains, and halogenated alkyl chains.

17. The capacitive energy storage system as in claim 13, wherein said metacapacitors are comprised of an oligomeric material described by the general formula: 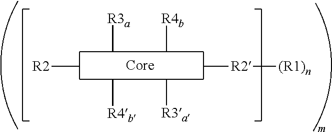
where Core is an aromatic polycyclic conjugated molecule, R1 is an organic substituent
that is soluble in organic solvents connected to the Core at terminal positions, lateral positions, and combinations thereof and is electrically resistive consisting of hydrocarbons, fluorocarbon, siloxane, polyethylene glycol, and mixtures thereof, n is an integer in the range of 0 to 8, R2 is an
electron donating substituent placed at terminal positions of Core and is
selected from the group consisting of NH₂, NR₂, NRR′, where R and R′ are groups consisting of —C₁X₂₁₊₁, —C(CX₃), and any combination thereof where X can be H, F, Cl, Br, and any combination thereof and 1 is an integer between 1 and 22, R3, R3′, R4, and R4′ are positioned at lateral
positions of Core and are independently selected from the list of ionic, hydrocarbon, haloalkyls, nitro, and amine substituents, and any combination thereof,
R3, R3′, R4, and R4′ are
independently connected to the Core structure by the group consisting of SP2 hybridized carbon bonds, SP3 hybridized carbon bonds, and a divalent connecting group, and a, a′, b, and b′ range between 0 and 4 and represent the number of R3, R3′, R4, and R4′ substituents respectively, and m is
an integer from 3 to 100,000 representing the number of aromatic polycyclic conjugated molecules in a supramolecular complex.

18. A capacitive energy storage system as in claim 17, wherein Core is comprised of repeating segments selected from the group consisting of rylene, phenylene, thiophene, polyacene quinine, and combinations thereof.

19. A capacitive energy storage system as in claim 17, wherein R1 is described by the formula C_{X}Q_{2X+1}, where X is ≧1 and Q is selected from the group consisting of hydrogen, fluorine, and chlorine.

20. A capacitive energy storage system as in claim 17, wherein R1 is selected from the group consisting of alkyl, aryl, fluorinated alkyl, chlorinated alkyl, branched alkyl, unsaturated alkyl, and combinations thereof.

21. A capacitive energy storage system as in claim 17, wherein R1 is selected from methyl, ethyl, propyl, butyl, iso-butyl, and tert-butyl.

22. A capacitive energy storage system as in claim 17, wherein R1 is selected from phenyl, benzyl, and naphthyl.

23. A capacitive energy storage system as in claim 17, wherein R1 is connected to Core by a connecting group selected from ether, amine, ester, amide, alkenyl, alkynyl, sulfonyl, sulfonate, and sulfonamide.

24. A capacitive energy storage system as in claim 17, wherein R2 is described by the formula C_{X}Q_{2X+1}, where X is ≧1 and Q is selected from the group consisting of hydrogen, fluorine, and chlorine.

25. A capacitive energy storage system as in claim 17, wherein R2 is selected from the group consisting of alkyl, aryl, fluorinated alkyl, chlorinated alkyl, branched alkyl, unsaturated alkyl, and combinations thereof.

26. A capacitive energy storage system as in claim 17, wherein R2 is selected from methyl, ethyl, propyl, butyl, iso-butyl, and tert-butyl.

27. A capacitive energy storage system as in claim 17, wherein R2 is selected from phenyl, benzyl, and naphthyl.

28. A capacitive energy storage system as in claim 17, wherein R2 is connected to Core by a connecting group selected from ether, amine, ester, amide, alkenyl, alkynyl, sulfonyl, sulfonate, and sulfonamide.

29. A capacitive energy storage system as in claim 17, wherein R3 and R4 are connected to Core by a connecting group independently selected from CH2, CF2, SiR2O, and CH2CH2O, wherein R is selected from hydrogen, alkyl, and fluorine.

30. A capacitive energy storage system as in claim 17, wherein R3 and R4 are independently selected from NR4+, PR4+, —CO2-, —SO3-, —SR5-, PO3R-, and —PR5-, —NO2, —NH3+ and —NR3+ (quaternary nitrogen salts), counterion Cl— or Br—, —CHO (aldehyde), —CRO (keto group), —SO3H (sulfonic acids), —SO3R (sulfonates), SO2NH2 (sulfonamides), —COOH (carboxylic acid), —COOR (esters, from carboxylic acid side), —COCl (carboxylic acid chlorides), —CONH2 (amides, from carboxylic acid side), —CF3, —CCl3, —CN, —O— (phenoxides, like —ONa or —OK), —NH2, —NHR, NR2, —OH, —OR (ethers), —NHCOR (amides, from amine side), —OCOR (esters, from alcohol side), alkyls, —C6H5, vinyls, wherein R is radical selected from the list comprising alkyl (methyl, ethyl, isopropyl, tert-butyl, neopentyl, cyclohexyl etc.), allyl (—CH2—CH═CH2), benzyl (—CH2C6H5) groups, phenyl (+substituted phenyl) and other aryl (aromatic) groups, hydrogen, and fluorine.

31. A capacitive energy storage system as in claim 13, wherein said metacapacitors are comprised of a polymeric material described by the general formula: 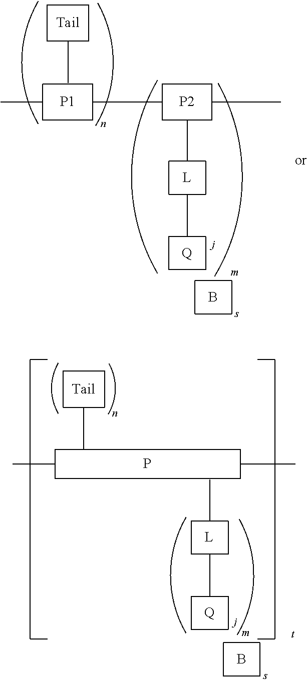
wherein P, P1, and P2 are randomly repeating polymeric units independently selected from (meth)acrylate, polypropylene, polyethylene, siloxane, and polyethylene terephthalate, n is the number of polymeric units bearing the Tail substituents and is an integer from 3 to 100,000, m is the number of polymeric units bearing L-Q substituents and is an integer from 3 to 100,000, Q is an ionic functional group connected to the polymeric backbone by linker group L, j is the number of Q groups attached to L and is an integer from 0 to 5, B is a counter ion of opposite charge of the polymer, s is the number of counter ions, and t is the average number of repeat units and is an integer from 6 to 200,000.

32. A capacitive energy storage system as in claim 31, wherein Tail is a resistive oligomer of polymeric material with a HOMO-LUMO gap of no less than 4 eV.

33. A capacitive energy storage system as in claim 31, wherein Tail is selected from the group consisting of hydrocarbon, fluorocarbon, siloxane, and polyethylene glycol.

34. A capacitive energy storage system as in claim 31, wherein Q is selected from the group consisting of ionic liquid ions, zwitterions, and polymeric acids.

35. A capacitive energy storage system as in claim 31, wherein Q has an energy interaction of less than kT, where k is the Boltzmann constant and T is the temperature of the environment.
